(19) United States
(12) Patent Application Publication
(10) Pub. No.: US 2010/0086929 A1
(43) Pub. Date: Apr. 8, 2010

(54) DETECTION OF ANALYTES

(75) Inventor: Eran Gabbai, Kfar-MaAs (IL)

Correspondence Address: FOLEY AND LARDNER LLP;SUITE 500, 3000 K STREET NW, WASHINGTON, DC, 20007, US

(73) Assignee: DO-COOP TECHNOLOGIES LTD., Or-yehuda (IL)

(21) Appl. No.: 12/521,847

(22) PCT Filed: Jan. 3, 2008
(86) PCT No.: PCT/IL08/00024

§ 371 Date: Jun. 30, 2009

Related U.S. Application Data
(60) Provisional application No. 60/878,388, filed on Jan. 4, 2007.

(51) Int. Cl. C12Q 1/68; G01N 33/53

(52) U.S. Cl. 435/6; 435/7.1

(57) ABSTRACT

A kit and article of manufacture for detecting an analyte is disclosed. The kit comprises:
(i) a detectable agent; and
(ii) a liquid composition having a liquid and nanostructures, each of the nanostructures comprising a core material of a nanometric size surrounded by an envelope of ordered fluid molecules, the core material and the envelope of ordered fluid molecules being in a steady physical state.

### FIELD AND BACKGROUND OF THE INVENTION

The present invention relates to kits and articles of manufacture which can be used to enhance the detection of an analyte.

The detection of biomolecules, for example proteins, can be highly beneficial in the diagnosis of diseases or medical conditions. By determining the presence of a specific protein or properties associated with a specific protein, investigators can confirm the presence of a virus, bacterium, genetic mutation, or other condition that relates to a disease-state. Furthermore, by analyzing a patient's proteome, i.e., the patient's unique set of expressed proteins, useful information relating to an individual's need for particular medicines or therapies can be determined, so as to customize a course of treatment or preventative therapy. Current methods for detecting proteins and peptides include simple methods such as Western blot analysis, Immunochemical assay, and enzyme-linked immunosorbent assay (ELISA).

Use of radiopharmaceuticals is generally the most common method for detecting biomolecules. However, the very success and widespread use of radioimmunoassays has raised several problems which include: (1) shelf-life and stability of radiolabeled compounds, (2) high cost of radioactive waste disposal, and (3) health hazards as a result of exposure to the use of not only radioactive materials but to the solvent necessary for liquid-scintillation counting, as well.

Compounds that fluoresce have many uses and are known to be particularly suitable for biological applications where fluorescence is intrinsically more sensitive than absorption as the incidence and observed wavelengths are different. Fluorescence can be used for the detection of whole cells, cellular components, and cellular functions. For example, many diagnostic and analytical techniques require the samples to be fluorescently tagged so that they can be detected. This is achieved by using fluorescent dyes or probes which interact with a wide variety of materials such as cells, tissues, proteins, antibodies, enzymes, drugs, hormones, lipids, nucleotides, nucleic acids, carbohydrates, or natural or synthetic polymers to make fluorescent conjugates.

With synthetic fluorescent probes, ligands are frequently used to confer a specificity for a biochemical reaction that is to be observed and the fluorescent dye provides the means of detect or quantify the interaction. These applications include, among others, the detection of proteins (for example in gels, on surfaces or aqueous solution), cell tracking, the assessment of enzymatic activity and the staining of nucleic acids or other biopolymers.

Chemiluminescence, i.e. the production of light by chemical reaction, and bioluminescence, i.e. the light produced by some living organisms, have been tested as potential replacements for radioactive labels, not only in protein detection, but also, DNA sequencing and other related research. Chemiluminescence provides a major advantage over radioactive labeling because it generates cold light i.e. its generated light is not caused by vibrations of atoms and/or molecules involved in the to reaction but by direct transformation of chemicals into electronic energy. Thus, research on the chemiluminescence of organic compounds is an on-going area of major emphasis. Parenthetically, chemiluminescence is also advantageous in detecting and measuring trace elements and pollutants for environmental control.

The best known chemiluminescent reactions are those which employ either stabilized enzmye triggerable 1,2-dioxetanes, acridanes, acridinium esters, luminol, isoluminol and derivatives thereof or lucigenin, as the chemical agent, reactant or substrate.

Horseradish peroxidase is widely used for assays because it is widely available and inexpensive to use. Horseradish peroxidase catalyzes the luminescent oxidation of a wide range of substrates including cyclic hydrazide, phenol derivatives, acridane derivatives and components of bioluminescent systems. Other suitable substrates, also, include: (a) luminol and related compounds, (b) pyrogallol, and purpurogallin (c) acridanecarboxylic acid derivatives (d) luciferins isolated from Pholas dactlus, and the firefly Photinus pyralis or Cypridina. These light producing reactions differ widely in their detection limits, specificity, reagent availability and magnitude and kinetics of light emission. This, of course, restricts their applicability.

Whilst a number of fluorescent and chemiluminescent substrates are known in the art, there is still a need to improve their signal intensity, their signal to background ratio and/or their stability.

### SUMMARY OF THE INVENTION

According to one aspect of the present invention there is provided a kit for detecting an analyte comprising (i) a detectable agent; and (ii) a liquid composition having a liquid and nanostructures, each of the nanostructures comprising a core material of a nanometric size surrounded by an envelope of ordered fluid molecules, the core material and the envelope of ordered fluid molecules being in a steady physical state.

According to another aspect of the present invention there is provided an article of manufacture comprising packaging material and a liquid composition identified for enhancing detection of a detectable moiety being contained within the packaging material, the liquid composition having a liquid and nanostructures, each of the nanostructures comprising a core material of a nanometric size surrounded by an envelope of ordered fluid molecules, the core material and the envelope of ordered fluid molecules being in a steady physical state.

According to another aspect of the present invention there is provided a method of dissolving or dispersing cephalosporin comprising contacting the cephalosporin with nanostructures and liquid under conditions which allow dispersion or dissolving of the substance, wherein said nanostructures comprise a core material of a nanometric size enveloped by ordered fluid molecules of said liquid, said core material and said envelope of ordered fluid molecules being in a steady physical state.

According to further features in preferred embodiments of the invention described below, the analyte is a biomolecule.

According to still further features in the described preferred embodiments, the biomolecule is selected from the group consisting of a polypeptide, a polynucleotide, a carbohydrate, a lipid and a combination thereof.

According to still further features in the described preferred embodiments, the detectable agent is non-directly detectable.

According to still further features in the described preferred embodiments, the non-directly detectable agent is a substrate for an enzymatic reaction capable of generating a detectable product.

According to still further features in the described preferred embodiments, the detectable agent is directly detectable.

According to still further features in the described preferred embodiments, the detectable agent comprises an affinity recognition moiety.

According to still further features in the described preferred embodiments, the affinity recognition moiety is selected from the group consisting of an avidin derivative, a polynucleotide and an antibody.

According to still further features in the described preferred embodiments, the directly detectable agent is selected from the group consisting of a phosphorescent agent, a chemiluminescent agent and a fluorescent agent.

According to still further features in the described preferred embodiments, the kit further comprises an enhancer of the enzymatic reaction.

According to still further features in the described preferred embodiments, the enhancer is selected from the group consisting of p-iodophenol, 3,4-dichlorophenol, p-hydroxycinnamic acid, 1,2,4-triazole, 3,3′,5,5′-tetramethyl-benzidine, phenol, 2-naphthol, 10-methylphenothiazine, cetyltrimethyl ammonium bromide, and mixtures thereof.

According to still further features in the described preferred embodiments, the kit further comprising an oxidizing agent.

According to still further features in the described preferred embodiments, the oxidizing agent is selected from the group consisting of hydrogen peroxide, urea hydrogen peroxide, sodium carbonate hydrogen peroxide, a perborate salt, potassium ferricyanide and Nitro blue tetrazolium (NBT).

According to still further features in the described preferred embodiments, the kit further comprises an enzyme for the enzymatic reaction.

According to still further features in the described preferred embodiments, the enzyme is selected from the group consisting of alkaline phosphatase, β-galactosidase, horseradish peroxidase (HRP), chloramphenicol acetyl transferase, luciferase and β-glucuronidase.

According to still further features in the described preferred embodiments, the enzyme is conjugated to an antibody or an avidin derivative.

According to still further features in the described preferred embodiments, the kit further comprises an inhibitor of the enzymatic reaction.

According to still further features in the described preferred embodiments, the detectable product is selected from the group consisting of a fluorescent product, a chemiluminescent product, a phosphorescent product and a chromogenic product.

According to still further features in the described preferred embodiments, a substrate capable of generating the fluorescent product comprises a fluorophore.

According to still further features in the described preferred embodiments, the fluorophore is derived from a molecule selected from the group consisting of coumarin, fluorescein, rhodamine, resorufin and DDAO.

According to still further features in the described preferred embodiments, a substrate capable of generating the fluorescent product is selected from the group consisting of fluorescein di-β-D-galactopyranoside (FDG), resorufin β-D-galactopyranoside, DDAO galactoside, β-methylumbelliferyl β-D-galactopyranoside, 6,8-Difluoro-4-methylumbelliferyl β-D-galactopyranoside, 3-carboxyumbelliferyl-β-D-galactopyranoside, ELF 97 phosphate, 5-chloromethylfluorescein galactopyranoside (CMFDG), 4-methylumbelliferyl-β-D-glucuronide, Fluorescein di-β-D-glucuronide, PFB Aminofluorescein Diglucuronide, ELF 9713-D-glucuronide, BODIPY FL chloramphenicol Substrate™, and 10-acetyl-3,7-dihydroxyphenoxazine.

According to still further features in the described preferred embodiments, a substrate capable of generating the chromogenic product is selected from the group consisting of BCIP, 5-bromo-4-chloro-3-indolyl-β-D-glucuronic acid (X-GlcU) and 5-bromo-6-chloro-3-indolyl-β-D-glucuronide, 5-bromo-4-chloro-3-indolyl-β-D-galactopyranoside (X-Gal), diaminobenzidine (DAB), Tetramethylbenzidine (TMB) and o-Phenylenediamine (OPD).

According to still further features in the described preferred embodiments, a substrate capable of generating the chemiluminescent product is selected from the group consisting of luciferin, luminol, isoluminol, acridane, phenyl-10-methylacridane-9-carboxylate, 2,4,6-trichlorophenyl-1-0-methylacridane-9-carboxylate, pyrogallol, phloroglucinol and resorcinol.

According to still further features in the described preferred embodiments, at least a portion of the fluid molecules are identical to molecule of said liquid.

According to still further features in the described preferred embodiments, the at least a portion of the fluid molecules are in a gaseous state.

According to still further features in the described preferred embodiments, a concentration of the nanostructures is lower than 10²⁰ nanostructures per liter.

According to still further features in the described preferred embodiments, the nanostructures are capable of forming clusters of the nanostructures.

According to still further features in the described preferred embodiments, the nanostructures are capable of maintaining long range interaction thereamongst.

According to still further features in the described preferred embodiments, the liquid composition comprises a buffering capacity greater than a buffering capacity of water.

According to still further features in the described preferred embodiments, the nanostructures are formulated from hydroxyapatite.

The present invention successfully addresses the shortcomings of the presently known configurations by providing compositions comprising enhanced capability for detecting an analyte.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-F is a photograph of an autoradiograph illustrating the increase in sensitivity of the ECL reaction using water comprising nanostructures. Cell lysates equivalent to 7.5 μg—strip (FIGS. 1A, 1C and 1E) and 15 μg strip (FIGS. 1B, 1D and 1F) of Jurkat cell line, were subjected to SDS-PAGE followed by protein blotting onto a nitrocellulose membrane. Following incubation with a polyclonal antibody raised against ZAP70, immunoreactive protein bands were visualized by reaction with HRP-conjugated secondary Ab and development with an immunoperoxidase ECL detection system. Lane 1—standard reaction reagents; Lane 2—all reagents+buffers using water comprising nanostructures; Lane 3—reaction volume made up with water comprising nanostructures.

FIG. 2 is a graph illustrating Sodium hydroxide titration of various water compositions as measured by absorbence at 557 nm.

FIGS. 3A-C are graphs of an experiment performed in triplicate illustrating Sodium hydroxide titration of water comprising nanostructures and RO water as measured by pH.

FIGS. 4A-C are graphs illustrating Sodium hydroxide titration of water comprising nanostructures and RO water as measured by pH, each graph summarizing 3 triplicate experiments.

FIGS. 5A-C are graphs of an experiment performed in triplicate illustrating Hydrochloric acid titration of water comprising nanostructures and RO water as measured by pH.

FIG. 6 is a graph illustrating Hydrochloric acid titration of water comprising nanostructures and RO water as measured by pH, the graph summarizing 3 triplicate experiments.

FIGS. 7A-C are graphs illustrating Hydrochloric acid (FIG. 10A) and Sodium hydroxide (FIGS. 10B-C) titration of water comprising nanostructures and RO water as measured by absorbence at 557 nm.

FIGS. 8A-B are photographs of cuvettes following Hydrochloric acid titration of RO (FIG. 8A) and water comprising nanostructures (FIG. 8B). Each cuvette illustrated addition of 1 μl of Hydrochloric acid.

FIGS. 9A-C are graphs illustrating Hydrochloric acid titration of RF water (FIG. 9A), RF2 water (FIG. 9B) and RO water (FIG. 9C). The arrows point to the second radiation.

FIG. 10 is a graph illustrating Hydrochloric acid titration of FR2 water as compared to RO water. The experiment was repeated three times. An average value for all three experiments was plotted for RO water.

FIGS. 11A-J are photographs of solutions comprising red powder and Neowater™ following three attempts at dispersion of the powder at various time intervals. FIGS. 11A-E illustrate right test tube C (50% EtOH+Neowater™) and left test tube B (dehydrated Neowater™) from Example 6 part C. FIGS. 11G-J illustrate solutions following overnight crushing of the red powder and titration of 100 μl Neowater™

FIGS. 12A-C are readouts of absorbance of 2 μl from 3 different solutions as measured in a nanodrop. FIG. 12A represents a solution of the red powder following overnight crushing+100 μl Neowater. FIG. 12B represents a solution of the red powder following addition of 100% dehydrated Neowater™ and FIG. 12C represents a solution of the red powder following addition of EtOH+Neowater™ (50%-50%).

FIG. 13 is a graph of spectrophotometer measurements of vial #1 (CD-Dau+Neowater™), vial #4 (CD-Dau+10% PEG in Neowater™) and vial #5 (CD-Dau+50% Acetone+50% Neowater™).

FIG. 14 is a graph of spectrophotometer measurements of the dissolved material in Neowater™ (blue line) and the dissolved material with a trace of the solvent acetone (pink line).

FIG. 15 is a graph of spectrophotometer measurements of the dissolved material in Neowater™ (blue line) and acetone (pink line). The pale blue and the yellow lines represent different percent of acetone evaporation and the purple line is the solution without acetone.

FIG. 16 is a graph of spectrophotometer measurements of CD-Dau at 200-800 nm. The blue line represents the dissolved material in RO while the pink line represents the dissolved material in Neowater™.

FIG. 17 is a graph of spectrophotometer measurements of t-boc at 200-800 nm. The blue line represents the dissolved material in RO while the pink line represents the dissolved material in Neowater™

FIGS. 18A-D are graphs of spectrophotometer measurements at 200-800 nm.

FIG. 18A is a graph of AG-14B in the presence and absence of ethanol immediately following ethanol evaporation. FIG. 18B is a graph of AG-14B in the presence and absence of ethanol 24 hours following ethanol evaporation. FIG. 18C is a graph of AG-14A in the presence and absence of ethanol immediately following ethanol evaporation. FIG. 18D is a graph of AG-14A in the presence and absence of ethanol 24 hours following ethanol evaporation.

FIG. 19 is a photograph of suspensions of AG-14A and AG14B 24 hours following evaporation of the ethanol.

FIGS. 20A-G are graphs of spectrophotometer measurements of the peptides dissolved in Neowater™. FIG. 20A is a graph of Peptide X dissolved in Neowater™. FIG. 20B is a graph of X-5FU dissolved in Neowater™. FIG. 20C is a graph of NLS-E dissolved in Neowater™. FIG. 20D is a graph of Palm-PFPSYK (CMFU) dissolved in Neowater™. FIG. 20E is a graph of PFPSYKLRPG-NH₂ dissolved in Neowater™. FIG. 20F is a graph of NLS-p2-LHRH dissolved in Neowater™, and FIG. 20G is a graph of F-LH-RH-palm kGFPSK dissolved in Neowater™.

FIGS. 21A-G are bar graphs illustrating the cytotoxic effects of the peptides dissolved in Neowater™ as measured by a crystal violet assay. FIG. 21A is a graph of the cytotoxic effect of Peptide X dissolved in Neowater™. FIG. 21B is a graph of the cytotoxic effect of X-5FU dissolved in Neowater™. FIG. 21C is a graph of the cytotoxic effect of NLS-E dissolved in Neowater™. FIG. 21D is a graph of the cytotoxic effect of Palm-PFPSYK (CMFU) dissolved in Neowater™. FIG. 21E is a graph of the cytotoxic effect of PFPSYKLRPG-NH₂ dissolved in Neowater™.

FIG. 21F is a graph of the cytotoxic effect of NLS-p2-LHRH dissolved in Neowater™, and FIG. 21G is a graph of the cytotoxic effect of F-LH-RH-palm kGFPSK dissolved in Neowater™

FIG. 22 is a graph of retinol absorbance in ethanol and Neowater™

FIG. 23 is a graph of retinol absorbance in ethanol and Neowater™ following filtration.

FIGS. 24A-B are photographs of test tubes, the left containing Neowater™ and substance “X” and the right containing DMSO and substance “X”. FIG. 24A illustrates test tubes that were left to stand for 24 hours and FIG. 24B illustrates test tubes that were left to stand for 48 hours.

FIGS. 25A-C are photographs of test tubes comprising substance “X” with solvents 1 and 2 (FIG. 28A), substance “X” with solvents 3 and 4 (FIG. 25B) and substance “X” with solvents 5 and 6 (FIG. 25C) immediately following the heating and shaking procedure.

FIGS. 26A-C are photographs of test tubes comprising substance “X” with solvents 1 and 2 (FIG. 26A), substance “X” with solvents 3 and 4 (FIG. 26B) and substance “X” with solvents 5 and 6 (FIG. 26C) 60 minutes following the heating and shaking procedure.

FIGS. 27A-C are photographs of test tubes comprising substance “X” with solvents 1 and 2 (FIG. 27A), substance “X” with solvents 3 and 4 (FIG. 27B) and substance “X” with solvents 5 and 6 (FIG. 27C) 120 minutes following the heating and shaking procedure.

FIGS. 28A-C are photographs of test tubes comprising substance “X” with solvents 1 and 2 (FIG. 28A), substance “X” with solvents 3 and 4 (FIG. 28B) and substance “X” with solvents 5 and 6 (FIG. 28C) 24 hours following the heating and shaking procedure.

FIGS. 29A-D are photographs of glass bottles comprising substance “X” in a solvent comprising Neowater™ and a reduced concentration of DMSO, immediately following shaking (FIG. 29A), 30 minutes following shaking (FIG. 29B), 60 minutes following shaking (FIG. 29C) and 120 minutes following shaking (FIG. 29D).

FIG. 30 is a graph illustrating the absorption characteristics of material “X” in RO/Neowater™ 6 hours following vortex, as measured by a spectrophotometer. FIGS. 31A-B are graphs illustrating the absorption characteristics of SPL2101 in ethanol (FIG. 31A) and SPL5217 in acetone (FIG. 31B), as measured by a spectrophotometer.

FIGS. 32A-B are graphs illustrating the absorption characteristics of SPL2101 in Neowater™ (FIG. 32A) and SPL5217 in Neowater™ (FIG. 32B), as measured by a spectrophotometer.

FIGS. 33A-B are graphs illustrating the absorption characteristics of taxol in Neowater™ (FIG. 33A) and DMSO (FIG. 33B), as measured by a spectrophotometer.

FIG. 34 is a bar graph illustrating the cytotoxic effect of taxol in different solvents on 293T cells. Control RO=medium made up with RO water; Control Neo=medium made up with Neowater™; Control DMSO RO=medium made up with RO water+10 μl DMSO; Control Neo RO=medium made up with RO water+10 μl Neowater™; Taxol DMSO RO=medium made up with RO water+taxol dissolved in DMSO; Taxol DMSO Neo=medium made up with Neowater™+taxol dissolved in DMSO; Taxol NW RO=medium made up with RO water+taxol dissolved in Neowater™; Taxol NW Neo=medium made up with Neowater™+taxol dissolved in Neowater™.

FIGS. 35A-B are photographs of a DNA gel stained with ethidium bromide illustrating the PCR products obtained in the presence and absence of the liquid composition comprising nanostructures following heating according to the protocol described in Example 14 using two different Taq polymerases.

FIG. 36 is a photograph of a DNA gel stained with ethidium bromide illustrating the PCR products obtained in the presence and absence of the liquid composition comprising nanostructures following heating according to the protocol described in Example 15 using two different Taq polymerases.

FIG. 37A is a graph illustrating the spectrophotometric readouts of 0.5 mM taxol in Neowater™ and in DMSO.

FIGS. 37B-C are HPLC readouts of taxol in Neowater™ and in DMSO. FIG. 37B illustrates the HPLC readout of a freshly prepared standard (DMSO) formulation of taxol. FIG. 37C illustrates the HPLC readout of taxol dispersed in Neowater™ after 6 months of storage at −20° C.

FIG. 38 is a bar graph illustrating PC3 cell viability of various taxol concentrations in DMSO or Neowater™ formulations. Each point represents the mean+/−standard deviation from eight replicates.

FIG. 39 is a spectrophotometer readout of cephalosporin dissolved in 100% acetone.

FIG. 40 is a spectrophotometer readout of Cephalosporin dissolved in Neowater™ prior to and following filtration.

FIGS. 41A-B are DH5α growth curves in LB with different Cephalosporin concentrations. Bacteria were grown at 37° C. and 220 rpm on two separate occasions.

FIGS. 42A-B are bar graphs illustrating DH5α viability with two different Cephalosporin concentrations in reference to the control growth (no Cephalosporin added) 7 h post inoculation on two separate occasions (the control group contains 100 μl of Neowater™).

### DESCRIPTION OF THE PREFERRED EMBODIMENTS

The present invention relates to kits and articles of manufacture which can be used to enhance the detection of an analyte.

The principles and operation of the kits and articles of manufacture according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The medical and diagnostic testing industries are constantly searching for more sensitive methods for detecting biomolecules. For example, medicine has an obvious need for highly sensitive methods of detecting viruses. More sensitive assays for the detection of chemicals or other substances would also be of use in a broad range of environmental areas, where early detection could trigger corrective action early enough to head off disaster. A highly sensitive detection technology could also be useful for the optimized control of semiconductor fabrication.

Whilst reducing the present invention to practice, the present inventors have uncovered that compositions comprising nanostructures (such as described in U.S. patent application No. 60/545,955 and Ser. No. 10/865,955, and International Patent Application, Publication No. WO2005/079153) enhance detection of an analyte.

As illustrated hereinbelow and in the Examples section which follows the present inventors have demonstrated that nanostructures and liquid increases the sensitivity of an ECL protein detecting system.

Thus, according to one aspect of the present invention there is provided an article of manufacture comprising packaging material and a liquid composition identified for enhancing detection of a detectable moiety being contained within the packaging material, the liquid composition having a liquid and nanostructures, each of the nanostructures comprising a core material of a nanometric size surrounded by an envelope of ordered fluid molecules, the core material and the envelope of ordered fluid molecules being in a steady physical state.

As used herein the term “nanostructure” refers to a structure on the sub-micrometer scale which includes one or more particles, each being on the nanometer or sub-nanometer scale and commonly abbreviated “nanoparticle”. The distance between different elements (e.g., nanoparticles, molecules) of the structure can be of order of several tens of picometers or less, in which case the nanostructure is referred to as a “continuous nanostructure”, or between several hundreds of picometers to several hundreds of nanometers, in which the nanostructure is referred to as a “discontinuous nanostructure”. Thus, the nanostructure of the present embodiments can comprise a nanoparticle, an arrangement of nanoparticles, or any arrangement of one or more nanoparticles and one or more molecules.

The liquid of the above-described composition is preferably an aquatic liquid e.g., water.

According to one preferred embodiment of this aspect of the present invention the nanostructures of the liquid composition comprise a core material of a nanometer size enveloped by ordered fluid molecules, which are in a steady physical state with the core material and with each other. Such a liquid composition is described in U.S. patent application No. 60/545,955 and Ser. No. 10/865,955 and International Pat. Appl. Publication No. WO2005/079153 to the present inventor, the contents of which are incorporated herein by reference.

Examples of such core materials include, without being limited to, a ferroelectric material, a ferromagnetic material and a piezoelectric material. A ferroelectric material is a material that maintains, over some temperature range, a permanent electric polarization that can be reversed or reoriented by the application of an electric field. A ferromagnetic material is a material that maintains permanent magnetization, which is reversible by applying a magnetic field. Preferably, the nanostructures retains the ferroelectric or ferromagnetic properties of the core material, thereby incorporating a particular feature in which macro scale physical properties are brought into a nanoscale environment.

The core material may also have a crystalline structure.

As used herein, the phrase “ordered fluid molecules” refers to an organized arrangement of fluid molecules which are interrelated, e.g., having correlations thereamongst. For example, instantaneous displacement of one fluid molecule can be correlated with instantaneous displacement of one or more other fluid molecules enveloping the core material.

As used herein, the phrase “steady physical state” is referred to a situation in which objects or molecules are bound by any potential having at least a local minimum. Representative examples, for such a potential include, without limitation, Van der Waals potential, Yukawa potential, Lenard-Jones potential and the like. Other forms of potentials are also contemplated.

Preferably, the ordered fluid molecules of the envelope are identical to the liquid molecules of the liquid composition. The fluid molecules of the envelope may comprise an additional fluid which is not identical to the liquid molecules of the liquid composition and as such the envelope may comprise a heterogeneous fluid composition.

Due to the formation of the envelope of ordered fluid molecules, the nanostructures of the present embodiment preferably have a specific gravity that is lower than or equal to the specific gravity of the liquid.

The fluid molecules may be either in a liquid state or in a gaseous state or a mixture of the two.

A preferred concentration of the nanostructures is below 10²⁰ nanostructures per liter and more preferably below 10¹⁵ nanostructures per liter. Preferably a nanostructure in the liquid is capable of clustering with at least one additional nanostructure due to attractive electrostatic forces between them. Preferably, even when the distance between the nanostructures prevents cluster formation (about 0.5-10 ?m), the nanostructures are capable of maintaining long-range interactions.

Without being bound to theory, it is believed that the long-range interactions between the nanostructures lends to the unique characteristics of the liquid composition such that it enhances the sensitivity of a detection system. For example, the present inventors have shown that the composition of the present invention shields and stabilizes proteins from the effects of heat—Examples 14 and 15; and comprises an enhanced buffering capacity (i.e. greater than the buffering capacity of water)—Examples 2-5. Both these factors may contribute to the state of proteins in the detection system, enhancing the overall sensitivity of the detection system.

As used herein, the phrase “buffering capacity” refers to the composition's ability to maintain a stable pH stable as acids or bases are added.

Furthermore, the present inventors have shown that the composition of the present invention enhances the solubility of agents Examples 6-13 and 15-17. This in turn may lead to an enhanced sensitivity of the detection system.

Production of the nanostructures according to this aspect of the present invention may be carried out using a “top-down” process. The process comprises the following method steps, in which a solid powder (e.g., a mineral, a ceramic powder, a glass powder, a metal powder, or a synthetic polymer) is heated, to a sufficiently high temperature, preferably more than about 700 ?C.

Examples of solid powders which are contemplated include, but are not limited to, BaTiO₃, WO₃ and Ba₂F₉O₁₂. Suprisingly, the present inventors have also shown that hydroxyapetite (HA) may also be heated to produce the liquid composition of the present invention. Hydroxyapatite is specifically preferred as it is characterized by intoxocicty and is generally FDA approved for human therapy.

It will be appreciated that many hydroxyapatite powders are available from a variety of manufacturers such as from Sigma Aldrich and Clarion Pharmaceuticals (e.g. Catalogue No. 1306-06-5).

As shown in Table 1, liquid compositions based on HA, all comprised enhanced buffering capacities as compared to water.

The heated powder is then immersed in a cold liquid, (water), below its density anomaly temperature, e.g., 3 ?C or 2 ?C. Simultaneously, the cold liquid and the powder are irradiated by electromagnetic RF radiation, preferably above 500 MHz, 700 MHz or more, which may be either continuous wave RF radiation or modulated RF radiation.

The present inventors have reasoned that the composition comprising nanostructures and liquid may increase the sensitivity of a detection system either by enhancement of the detectable signal and/or by increasing the activity of an enzyme responsible for the generation of such a signal.

It will be appreciated that the composition comprising nanostructures and liquid described hereinabove can form a part of a kit.

Thus, according to another aspect of the present invention there is provided a kit for detecting an analyte comprising:

(i) a detectable agent; and

(ii) a liquid composition having a liquid and nanostructures, each of the nanostructures comprising a core material of a nanometric size surrounded by an envelope of ordered fluid molecules, the core material and the envelope of ordered fluid molecules being in a steady physical state.

The kits of the present invention may, if desired, be presented in a pack which may contain one or more units of the kit of the present invention. The pack may be accompanied by instructions for using the kit. The pack may also be accommodated by a notice associated with the container in a form prescribed by a governmental agency regulating the manufacture, use or sale of laboratory supplements, which notice is reflective of approval by the agency of the form of the compositions.

As used herein, the term “analyte” refers to a molecule or compound to be detected. Suitable analytes include organic and inorganic molecules, including biomolecules. The analyte may be an environmental or clinical chemical or pollutant or biomolecule, including, but not limited to, pesticides, insecticides, toxins, therapeutic and abused drugs, hormones, antibiotics, organic materials, and solvents. Suitable biomolecules include, but are not limited to, polypeptides, polynucleotides, lipids, carbohydrates, steroids, whole cells [including prokaryotic (such as pathogenic bacteria) and eukaryotic cells, including mammalian tumor cells], viruses, spores, etc. Particularly preferred analytes are proteins including enzymes; drugs, antibodies; antigens; cellular membrane antigens and receptors (neural, hormonal, nutrient, and cell surface receptors) or their ligands.

The detection kits of the present invention show enhanced sensitivity by virtue of a liquid composition comprising liquid and nanostructures.

The present invention envisages solubilizing at least one component required for detection in the composition comprising liquid and nanostructures and/or performing the detection assay, wherein the water component is at least partly exchanged for the composition comprising liquid and nanostructures. The liquid portion of the detection assay may comprise 5%, more preferably 10%, more preferably 20%, more preferably 40%, more preferably 60%, more preferably 80% and even more preferably 100% of the liquid composition of the present invention.

As well as comprising a composition comprising liquid and nanostructures, the kits of the present invention also comprise a detectable agent.

According to one embodiment of this aspect of the present invention, the detectable agent is directly detectable typically by virtue of its emission of radiation of a particular wavelength (e.g. a fluorescent agent, phosphorescent agent or a chemiluminescent agent).

In order to detect a specific analyte, typically such detectable agents comprise affinity recognition moieties which bind to the target analyte. Examples of affinity recognition moieties include, but are not limited to avidin derivatives (e.g. avidin, strepavidin and nutravidin), antibodies and polynucleotides.

Avidin is a highly cationic 66,000-dalton glycoprotein with an isoelectric point of about 10.5. Streptavidin is a nonglycosylated 52,800-dalton protein with a near-neutral isoelectric point. Nutravidin is a deglycosylated form of avidin. All of these proteins have a very high affinity and selectivity for biotin, each capable of binding four biotins per molecule. A detectable agent comprising an avidin recognition moiety may be used for detecting naturally occurring biotinylated biomolecules, or biomolecules that have been artificially manipulated to comprise biotin.

The term “antibody” as used in this invention includes intact molecules as well as functional fragments thereof, such as Fab, F(ab′)₂, and Fv that are capable of binding to specific proteins or polypeptides.

The term “polynucleotide” as used herein, refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly to respective naturally-occurring portions. Labeled polynucleotides may be used to detect polynucleotides in a sample that are capable of hybridizing thereto.

As used herein, the phrase “capable of hybridizing” refers to base-pairing, where at least one strand of the nucleic acid agent is at least partly homologous to H19 mRNA.

According to another embodiment of this aspect of the present invention, the detectable agent of the kit of the present invention may also be non-directly detectable. For example, the detectable agent may be a substrate for an enzymatic reaction which is capable of generating a detectable product.

Substrates capable of generating a fluorescent product typically comprise fluorophores. Such fluorophores may be derived from many molecules including but not limited to coumarin, fluorescein, rhodamine, resorufin and DDAO.

Examples of substrates which are capable of generating a fluorescent product include, but are not limited to substrates yielding soluble fluorescent products (e.g. substrates derived from water-soluble coumarins, substrates derived from water-soluble green to yellow fluorophores, substrates derived from water-soluble red fluorophores, thiol-reactive fluorogenic substrates, lipophilic fluorophores, pentafluorobenzoyl fluorogenic enzyme substrate); substrates yielding insoluble fluorescent products, substrates based on excited-state energy transfer and fluorescent derivatization reagents for discontinuous enzyme assays). Details regarding such substrates may be found on the Invitrogen website (e.g. http://probes.invitrogen.com/handbook/sections/1001.html).

Specific examples of substrates capable of generating a fluorescent product include, but are not limited to fluorescein di-β-D-galactopyranoside (FDG), resorufin β-D-galactopyranoside, DDAO galactoside, β-methylumbelliferyl β-D-galactopyranoside, 6,8-Difluoro-4-methylumbelliferyl β-D-galactopyranoside, 3-carboxyumbelliferyl-β-D-galactopyranoside, ELF 97 phosphate, 5-chloromethylfluorescein di-β-D-galactopyranoside (CMFDG), 4-methylumbelliferyl-β-D-glucuronide, Fluorescein di-β-D-glucuronide, PFB Aminofluorescein Diglucuronide, ELF 97-β-D-glucuronide, BODIPY FL chloramphenicol Substrate™, and 10-acetyl-3,7-dihydroxyphenoxazine.

Examples of substrates capable of generating a chemiluminescent product include, but are not limited to luciferin, luminol, isoluminol, acridane, phenyl-10-methylacridane-9-carboxylate, 2,4,6-trichlorophenyl-1-0-methylacridane-9-carboxylate, pyrogallol, phloroglucinol and resorcinol.

Examples of substrates capable of generating a chromogenic product include, but are not limited to BCIP, 5-bromo-4-chloro-3-indolyl-β-D-glucuronic acid (X-GlcU) and 5-bromo-6-chloro-3-indolyl-β-D-glucuronide, 5-bromo-4-chloro-3-indolyl-β-D-galactopyranoside (X-Gal), diaminobenzidine (DAB), Tetramethylbenzidine (TMB) and o-Phenylenediamine (OPD).

The kits may be useful in a variety of detection assays.

Following is a list of assays for the detection of polynucleotides, which may be effected using the kits of the present invention.

Northern Blot analysis: This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using enzyme linked nucleotides. Detection may be effected using colorimetric reaction or chemiluminescence. This method allows both quantitation of an amount of particular RNA molecules and determination of its identity by a relative position on the membrane which is indicative of a migration distance in the gel during electrophoresis.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the slide is subjected to either a photographic emulsion which reveals signals generated using chemiluminescence associated probes or to a colorimetric reaction which reveals signals generated using enzyme-linked labeled probes.

Oligonucleotide microarray—In this method oligonucleotide probes capable of specifically hybridizing with the polynucleotides of the present invention are attached to a solid surface (e.g., a glass wafer). Each oligonucleotide probe is of approximately 20-25 nucleic acids in length. To detect the expression pattern of the polynucleotides of the present invention in a specific cell sample (e.g., blood cells), RNA is extracted from the cell sample using methods known in the art (using e.g., a TRIZOL solution, Gibco BRL, USA). Hybridization can take place using either labeled oligonucleotide probes (e.g., 5′-biotinylated probes) or labeled fragments of complementary DNA (cDNA) or RNA (cRNA). Briefly, double stranded cDNA is prepared from the RNA using reverse transcriptase (RT) (e.g., Superscript II RT), DNA ligase and DNA polymerase I, all according to manufacturer's instructions (Invitrogen Life Technologies, Frederick, Md., USA). To prepare labeled cRNA, the double stranded cDNA is subjected to an in vitro transcription reaction in the presence of biotinylated nucleotides using e.g., the BioArray High Yield RNA Transcript Labeling Kit (Enzo, Diagnostics, Affymetrix Santa Clara Calif.). For efficient hybridization the labeled cRNA can be fragmented by incubating the RNA in 40 mM Tris Acetate (pH 8.1), 100 mM potassium acetate and 30 mM magnesium acetate for 35 minutes at 94 ?C. Following hybridization, the microarray is washed and the hybridization signal is scanned using a confocal laser fluorescence scanner which measures fluorescence intensity emitted by the labeled cRNA bound to the probe arrays.

For example, in the Affymetrix microarray (Affymetrix®, Santa Clara, Calif.) each gene on the array is represented by a series of different oligonucleotide probes, of which, each probe pair consists of a perfect match oligonucleotide and a mismatch oligonucleotide. While the perfect match probe has a sequence exactly complimentary to the particular gene, thus enabling the measurement of the level of expression of the particular gene, the mismatch probe differs from the perfect match probe by a single base substitution at the center base position. The hybridization signal is scanned using the Agilent scanner, and the Microarray Suite software subtracts the non-specific signal resulting from the mismatch probe from the signal resulting from the perfect match probe.

Following is a list of assays for the detection of polypeptides, which may be effected using the kits of the present invention.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

In situ activity assay: According to this method, a chromogenic substrate is applied on the cells containing an active enzyme and the enzyme catalyzes a reaction in which the substrate is decomposed to produce a chromogenic product visible by a light or a fluorescent microscope.

According to one aspect of the present invention, the kits may be used to detect immobilized polypeptides or polynucleotides using a chemiluminescent detection assay.

In this assay, the target analyte is bound either directly or indirectly to an enzyme (e.g. horseradish peroxidase) which in the presence of an oxidizing agent is capable of catalyzing the oxidation of chemiluminescent substrates. Following oxidation the substrates are in an excited state and emit detectable light waves. Strong enhancement of the light emission may be produced by enhancers.

Accordingly, such kits may comprise, in addition to the liquid composition of the present invention and the detectable agent (i.e. chemiluminescent compounds such as luminol and those described hereinabove) enzymes capable of oxidizing the chemiluminescent substrates. Typically the enzyme is conjugated to an antibody or an avidin derivative such as strepavidin. Examples of such enzymes include, but are not limited to horseradish peroxidase, glucose oxidase, cholesterol oxidase and catalase.

The kits according to this aspect of the present invention may also comprise an oxidant. Exemplary oxidizing agents include hydrogen peroxide, urea hydrogen peroxide, sodium carbonate hydrogen peroxide or a perborate salt. Other oxidants or oxidizing agents known to those skilled in the art may be used herein. The preferred oxidant is either hydrogen peroxide or urea hydrogen peroxide and mixtures thereof.

As noted above, the kits of this aspect of the present invention may, also, include a chemiluminescence enhancer. Generally, the enhancer used herein comprises an organic compound which is soluble in an organic solvent or in a buffer and which enhances the luminescent reaction between the chemiluminescent organic compound, the oxidant and the enzyme or other biological molecule. Suitable enhancers include, for example, halogenated phenols, such as p-iodophenol, p-bromophenol, p-chlorophenol, 4-bromo-2-chlorophenol, 3,4-dichlorophenol, alkylated phenols, such as 4-methylphenol and, 4-tert-butylphenol, 3-(4-hydroxyphenyl)propionate and the like, 4-benzylphenol, 4-(2′,4′-dinitrostyryl) phenol, 2,4-dichlorophenol, p-hydroxycinnamic acid, p-fluorocinnamic acid, p-nitrocinnamic acid, p-aminocinnamic acid, m-hydroxycinnamic acid, o-hydroxycinnamic acid, 4-phenoxyphenol, 4-(4-hydroxyphenoxy)phenol, p-phenylphenol, 2-chloro-4-phenylphenol, 4′-(4′-hydroxyphenyl)benzophenone, 4-(phenylazo) phenol, 4-(2′-carboxyphenylaza)phenol, 1,6-dibromonaphtho-2-ol, 1-bromonaphtho-2-ol, 2-naphthol, 6-bromonaphth-2-ol, 6-hydroxybenzothiazole, 2-amino-6-hydroxybenzothiazol-e, 2,6-dihydroxybenzothiazole, 2-cyano-6-hydroxybenzothiazole, dehydroluciferin, firefly luciferin, phenolindophenol, 2,6-dichlorophenolindophenol, 2,6-dichlorophenol-o-cresol, phenolindoaniline, N-alkylphenoxazine or substituted N-alkylphenoxazine, N-alkylphenothiazine or substituted N-alkylphenothiazine, N-alkylpyrimidyl-phenoxazine or substituted N-alkylpyrimidylphenoxazine, N-alkylpyridylphenoxazine, 2-hydroxy-9-fluorenone or substituted 2-hydroxy-9-fluorenone, 6-hydroxybenzoxazole or substituted 6-hydroxybenzoxazole. Still other useful compounds include a protected enhancer that can be cleaved by the enzyme such as p-phenylphenol phosphate or p-iodophenol phosphate or other phenolic phosphates having other enzyme cleavable groups, as well as p-phenylene diamine and tetramethyl benzidine. Other useful enhancers include fluorescein, such as 5-(n-tetradecanyl) amino fluorescein and the like.

According to another aspect of the present invention, the kits may be used to detect immobilized polypeptides or polynucleotides using a fluorescent or chromogenic detection assay. Instead of comprising horseradish peroxidase or a derivate thereof, such kits typically comprise alkaline phosphatase and a fluorescent or choromogenic substrate. Oxidising agents for the production of chromogenic products may also be included in the kits such as potassium ferricyanide and Nitro blue tetrazolium (NBT).

The kits of the present invention may also be used for detecting the expression of several common reporter genes in cells and cell extracts. Thus the kits may comprise substrates for β-galactosidase β-glucuronidase, secreted alkaline phosphatase, chloramphenicol acetyltransferase and luciferase.

The kits of the present invention may further include inhibitors for the enzymatic reactions. Examples of such inhibitors include, but are not limited to levamisole, L-p-bromotetramisole, tetramisole and 5,6-Dihydro-6-(2-naphthyl)imidazo-[2,1-b]thiazole.

According to another aspect of the present invention there is provided a method of dissolving or dispersing cephalosporin, comprising contacting the cephalosporin with nanostructures and liquid under conditions which allow dispersion or dissolving of the substance, wherein the nanostructures comprise a core material of a nanometric size enveloped by ordered fluid molecules of the liquid, the core material and the envelope of ordered fluid molecules being in a steady physical state.

The cephalosporin may be dissolved in a solvent prior or following addition of the liquid composition of the present invention in order to aid in the solubilizing process. It will be appreciated that the present invention contemplates the use of any solvent including polar, non-polar, organic, (such as ethanol or acetone) or non-organic to further increase the solubility of the substance.

The solvent may be removed (completely or partially) at any time during the solubilizing process so that the substance remains dissolved/dispersed in the liquid composition of the present invention. Methods of removing solvents are known in the art such as evaporation (i.e. by heating or applying pressure) or any other method.

Additional objects, advantages, and novel features of the present invention will become apparent to one ordinarily skilled in the art upon examination of the to following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Effect of Water Comprising Nanostructures on an ECL Detection System

In order to determine if the sensitivity of an electrochemiluminescent reaction is affected by water comprising nanostructures, an HRP-conjugated secondary antibody was detected using an immunoperoxidase ECL detection system in the presence and absence of the above mentioned water.

Materials and Methods

Preparation of ECL Reagents: Stock A

a) 50 μl of 250 mM Luminol (Sigma C-9008) in DMSO (Fluca 0-9253).

b) 22 μl of 90 mM p-Coumaric acid (Sigma C-9008) in DMSO.

c) 0.5 ml Tris 1M, pH 8.5.

d) 4.428 ml H₂O (total of 5 ml).

### Stock B

a) 3 μl H₂O₂.

b) 0.5 ml Tris 1M, pH 8.5.

c) 4.5 ml H₂O (total of 5 ml).

Three different sources of ECL reagents were used.
1. Standard. Home made
2. Ver 1.0 The dH₂O was replaced for all the reagents and buffers with water comprising nanostructures.
3. Ver 1.1—The dH₂O of the reaction volume was replaced with water comprising nanostructures.

Whole cell protein extract was generated from Jurkat cells. The protein extract was subjected to SDS-PAGE followed by protein blotting onto a nitrocellulose membrane. An antibody specific for ZAP70 protein (home made polyclonal serum Ab) was incubated with the membrane at a dilution of 1:30000 (regular working dilution 1:3000). The antibody immunoreactive protein bands were visualized by reaction with HRP-conjugated secondary antibody followed by development with an immunoperoxidase ECL detection system. Essentially, an equal volume of stock A and stock B were combined and the detection mix was equilibrated for 5 minutes. The detection mix was added directly to the blot (protein side up) and incubated 3 minutes at room temperature. An x-ray film was then exposed to the nitrocellulose membrane for 1 minute, 5 minutes and 10 minutes.

Results

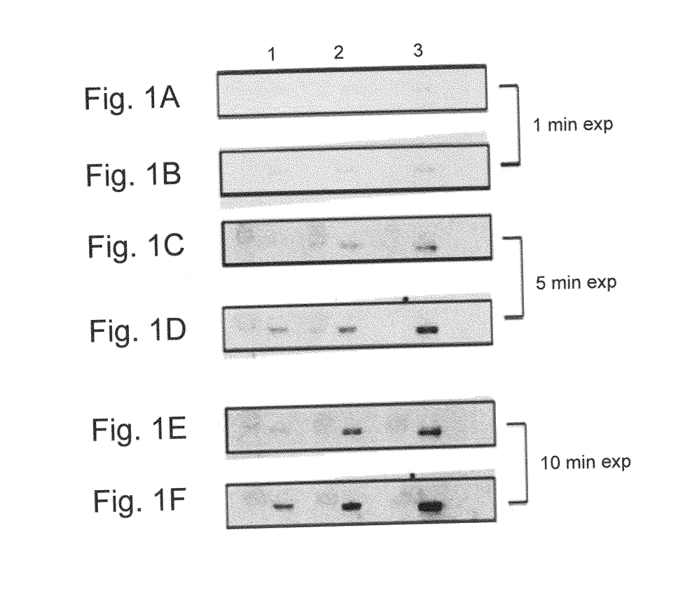

As illustrated in FIG. 1, replacing the water with water comprising nanostructures increases the sensitivity of the ECL reaction.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

### Example 2

### Buffering Capacity of the Composition Comprising Nanostructures

The effect of the composition comprising nanostructures on buffering capacity was examined.

Materials and Methods

Phenol red solution (20 mg/25 ml) was prepared. 290 μl was added to 13 ml RO water or various batches of water comprising nanostructures (Neowater™—Do-Coop technologies, Israel). It was noted that each water had a different starting pH, but all of them were acidic, due to their yellow or light orange color after phenol red solution was added. 2.5 ml of each water+phenol red solution were added to a cuvette. Increasing volumes of Sodium hydroxide were added to each cuvette, and absorption spectrum was read in a spectrophotometer. Acidic solutions give a peak at 430 nm, and alkaline solutions give a peak at 557 nm. Range of wavelength is 200-800 nm, but the graph refers to a wavelength of 557 nm alone, in relation to addition of 0.02M Sodium hydroxide.

Results

Table 1 summarizes the absorbance at 557 nm of each water solution following sodium hydroxide titration.

TABLE 1

| | | | | | | | μl of 0.02 |
|---|---|---|---|---|---|---|---|
| NW 1 | NW 2 | NW 3 | NW 4 | NW 5 | | | M sodium |
| HAP | AB 1-2-3 | HA 18 | Alexander | HA-99-X | NW 6 | RO | hydroxide added |
| 0.026 | 0.033 | 0.028 | 0.093 | 0.011 | 0.118 | 0.011 | 0 |
| 0.132 | 0.17 | 0.14 | 0.284 | 0.095 | 0.318 | 0.022 | 4 |
| 0.192 | 0.308 | 0.185 | 0.375 | 0.158 | 0.571 | 0.091 | 6 |
| 0.367 | 0.391 | 0.34 | 0.627 | 0.408 | 0.811 | 0.375 | 8 |
| 0.621 | 0.661 | 0.635 | 1.036 | 0.945 | 1.373 | 0.851 | 10 |
| 1.074 | 1.321 | 1.076 | 1.433 | 1.584 | 1.659 | 1.491 | 12 |

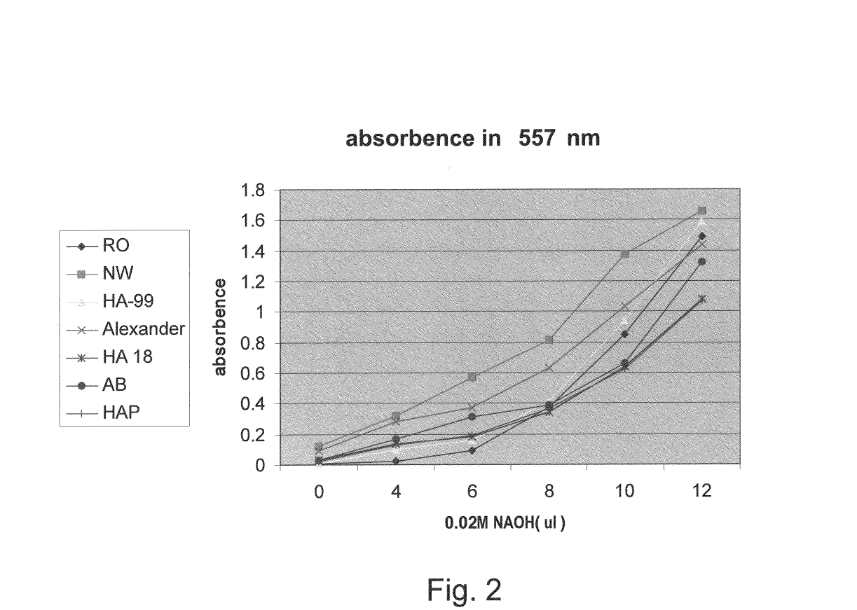

As illustrated in FIG. 2 and Table 2, RO water shows a greater change in pH when adding Sodium hydroxide. It has a slight buffering effect, but when absorbance reaches 0.09 A, the buffering effect “breaks”, and pH change is greater following addition of more Sodium hydroxide. HA-99 water is similar to RO. NW (#150905-106) (Neowater™), AB water Alexander (AB 1-22-1 HA Alexander) has some buffering effect. HAP and HA-18 shows even greater buffering effect than Neowater.

In summary, from this experiment, all new water types comprising nanostructures tested (HAP, AB 1-2-3, HA-18, Alexander) shows similar characters to Neowater™, except HA-99-X.

### Example 3

### Buffering Capacity of the Liquid Composition Comprising Nanostructures

The effect of the liquid composition comprising nanostructures on buffering capacity was examined.

Materials and Methods

Sodium hydroxide and Hydrochloric acid were added to either 50 ml of RO water or water comprising nanostructures (Neowater™—Do-Coop technologies, Israel) and the pH was measured. The experiment was performed in triplicate. In all, 3 experiments were performed.

Sodium hydroxide titration: −1 μl to 15 μl of 1M Sodium hydroxide was added.

Hydrochloric acid titration: −1 μl to 15 μl of 1M Hydrochloric acid was added.

Results

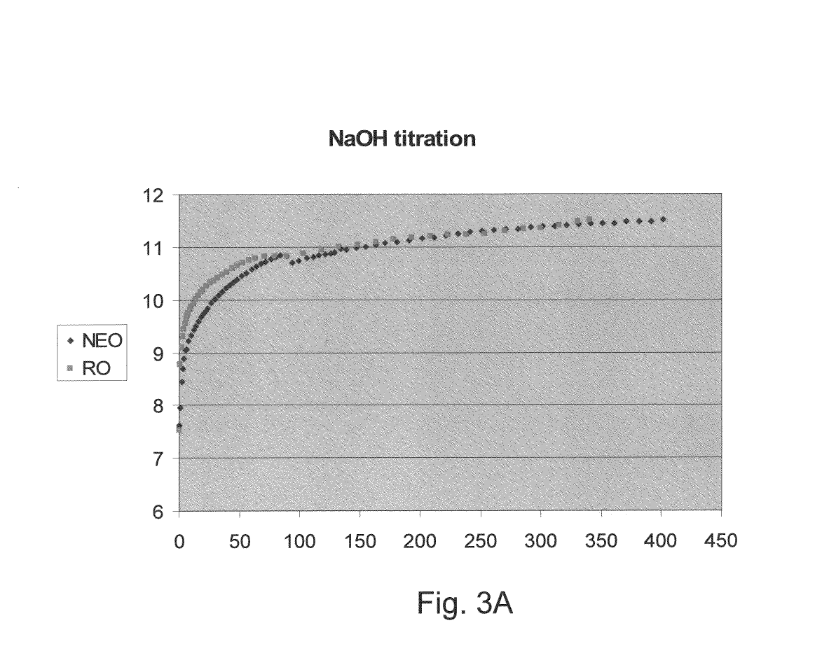

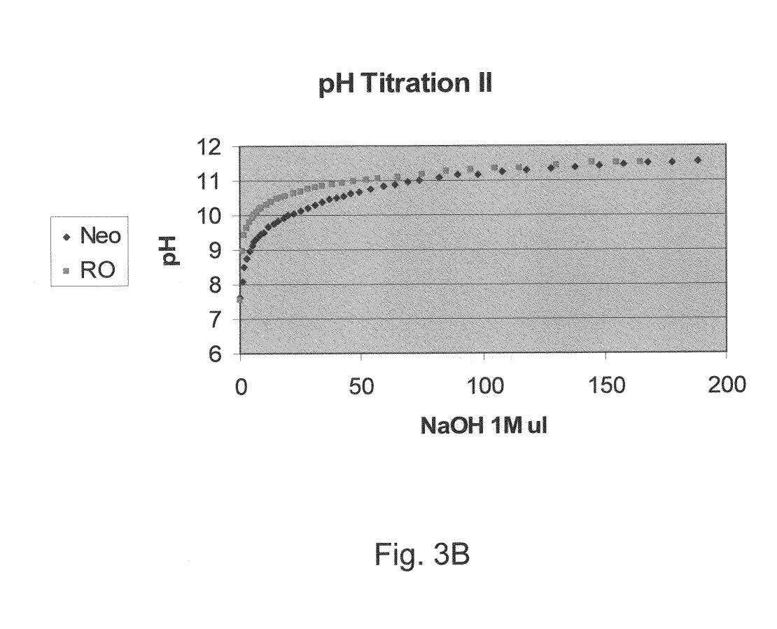

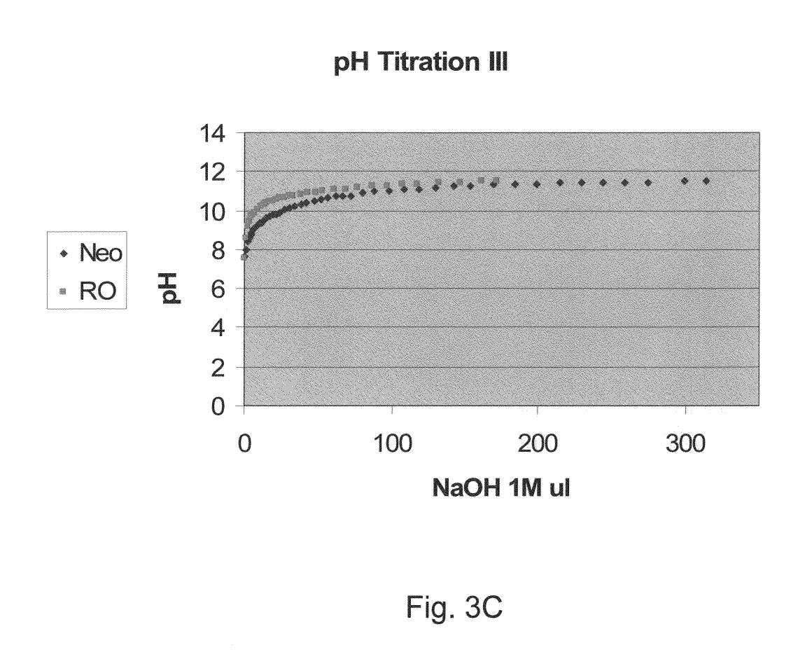

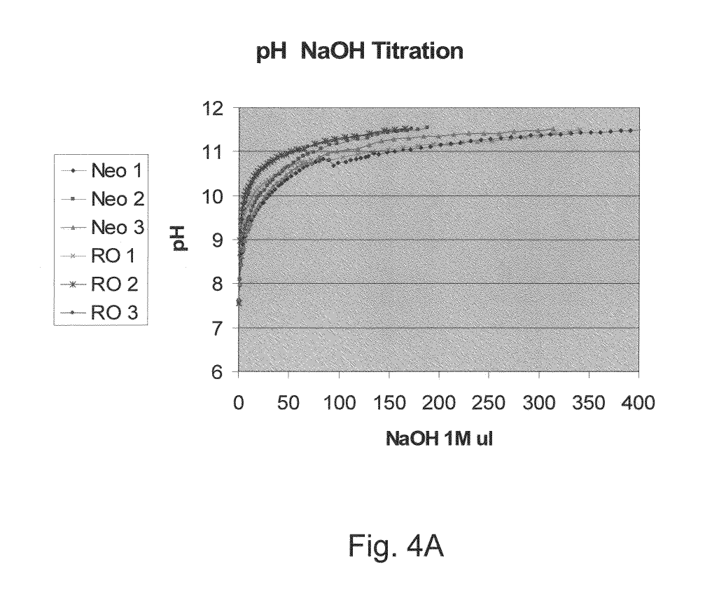

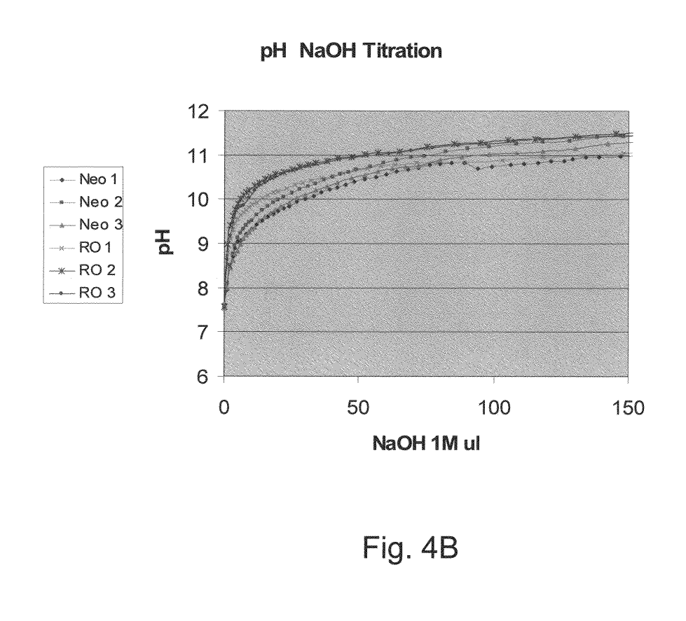

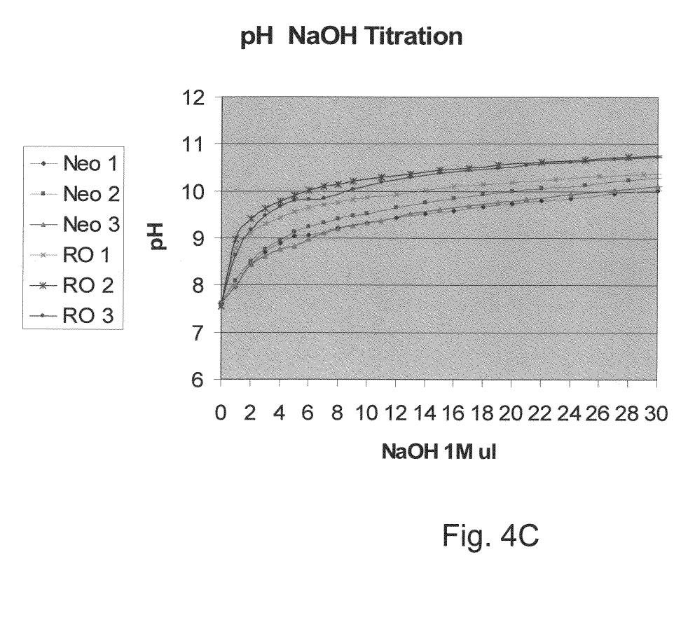

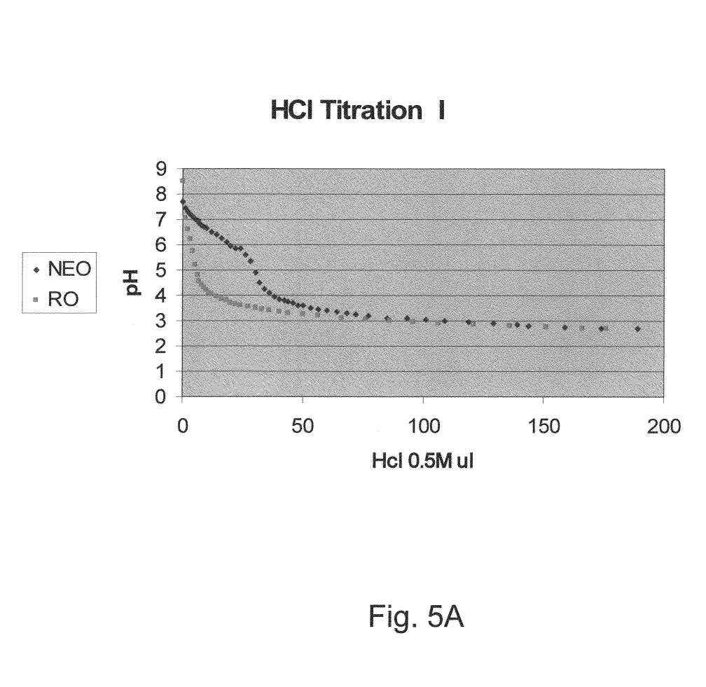

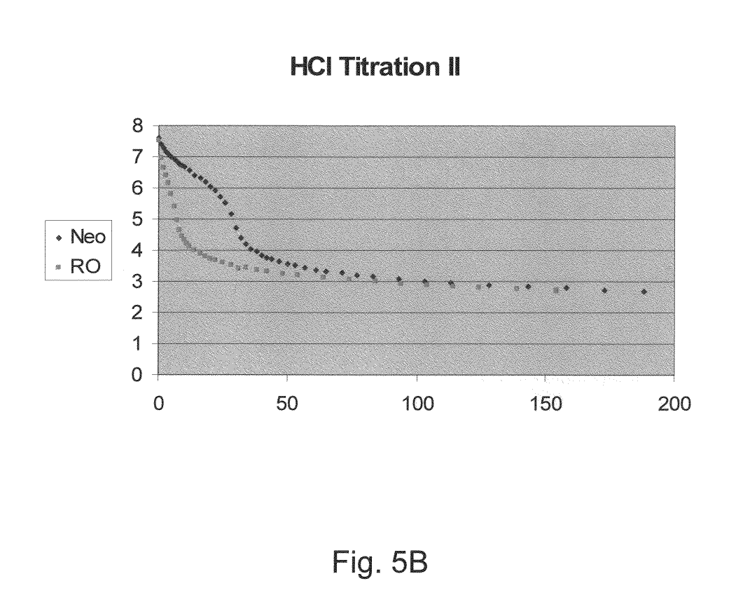

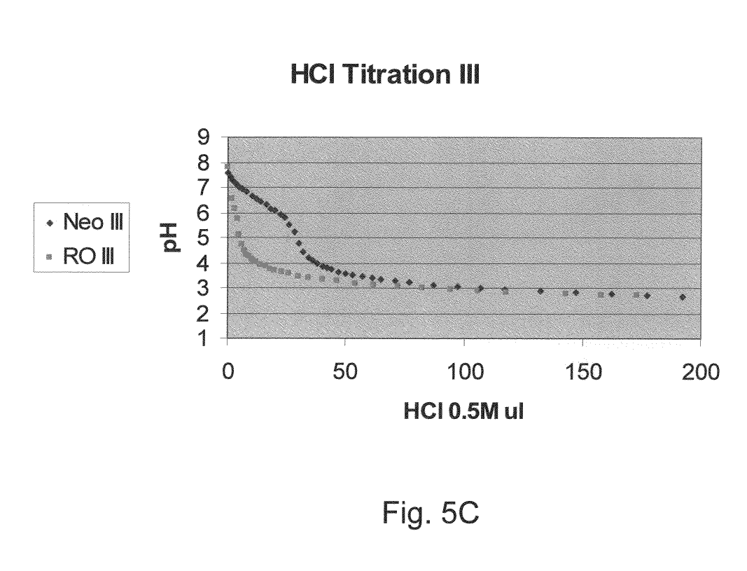

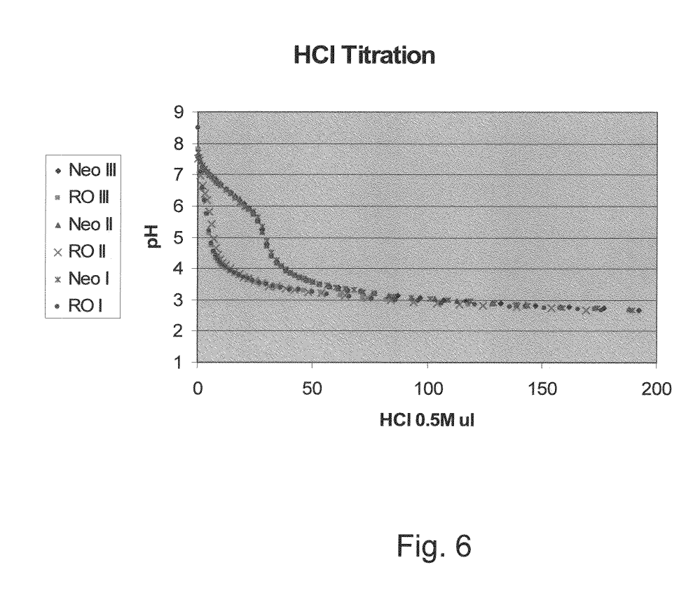

The results for the Sodium hydroxide titration are illustrated in FIGS. 3A-C and 4A-C. The results for the Hydrochloric acid titration are illustrated in FIGS. 5A-C and FIG. 6.

The water comprising nanostructures has buffering capacities since it requires greater amounts of Sodium hydroxide in order to reach the same pH level that is needed for RO water. This characterization is more significant in the pH range of 7.6-10.5. In addition, the water comprising nanostructures requires greater amounts of Hydrochloric acid in order to reach the same pH level that is needed for RO water. This effect is higher in the acidic pH range, than the alkali range. For example: when adding 10 μl Sodium hydroxide 1M (in a total sum) the pH of RO rises from 7.56 to 10.3. The pH of the water comprising nanostructures rose from 7.62 to 9.33. When adding 10 μl Hydrochloric acid 0.5M (in a total sum) the pH of RO decreased from 7.52 to 4.31 The pH of water comprising nanostructures decreased from 7.71 to 6.65. This characterization is more significant in the pH range of −7.7-3.

### Example 4

### Buffering Capacity of the Liquid Composition Comprising Nanostructures

The effect of the liquid composition comprising nanostructures on buffering capacity was examined.

Materials and Methods

Phenol red solution (20 mg/25 ml) was prepared. 1 ml was added to 45 ml RO water or water comprising nanostructures (Neowater™—Do-Coop technologies, Israel). pH was measured and titrated if required. 3 ml of each water+phenol red solution were added to a cuvette. Increasing volumes of Sodium hydroxide or Hydrochloric acid were added to each cuvette, and absorption spectrum was read in a spectrophotometer. Acidic solutions give a peak at 430 nm, and alkaline solutions give a peak at 557 nm. Range of wavelength is 200-800 nm, but the graph refers to a wavelength of 557 nm alone, in relation to addition of 0.02M Sodium hydroxide.

Hydrochloric Acid Titration:

### RO: 45 ml pH 5.8

1 ml phenol red and 5 μl Sodium hydroxide 1M was added, new pH=7.85 Neowater™ (# 150905-106): 45 ml pH 6.3

1 ml phenol red and 4 μl Sodium hydroxide 1M was added, new pH=7.19

Sodium Hydroxide Titration:

### I. RO: 45 ml pH 5.78

1 ml phenol red, 6 μl Hydrochloric acid 0.25M and 4 μl Sodium hydroxide 0.5M was added, new pH=4.43

### Neowater™ (# 150604-109): 45 ml pH 8.8

1 ml phenol red and 45 μl Hydrochloric acid 0.25M was added, new pH=4.43

### II. RO: 45 ml pH 5.78

1 ml phenol red and 5 μl Sodium hydroxide 0.5M was added, new pH=6.46

### Neowater™ (ft 120104-107): 45 ml pH 8.68

1 ml phenol red and 5 μl Hydrochloric acid 0.5M was added, new pH=6.91

Results

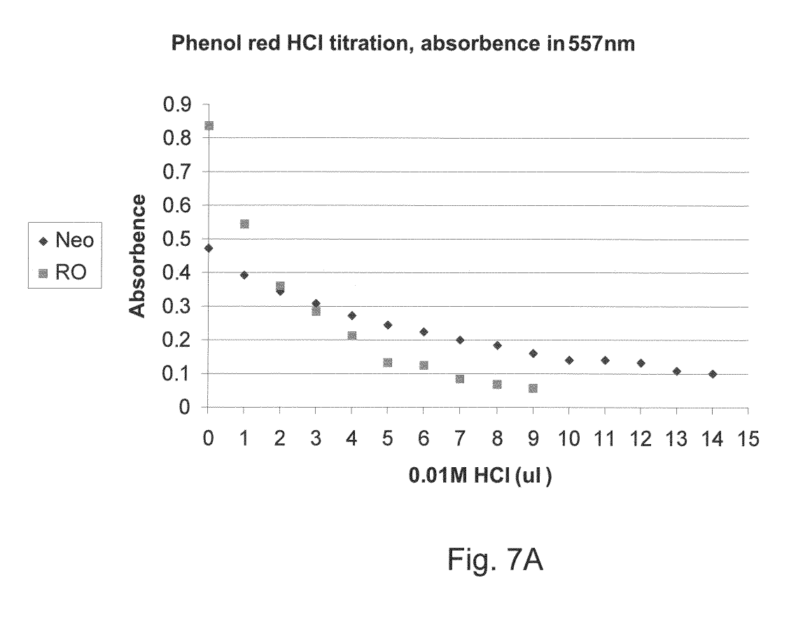

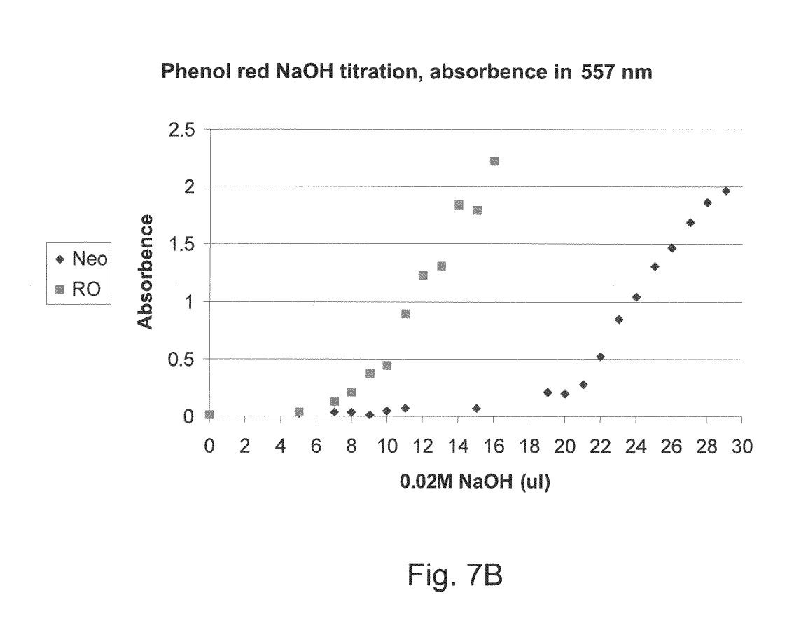

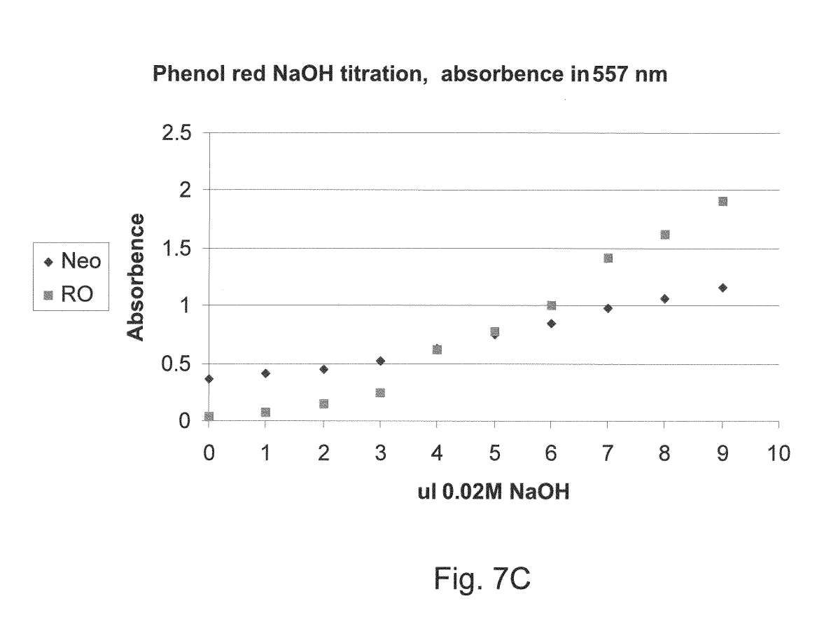

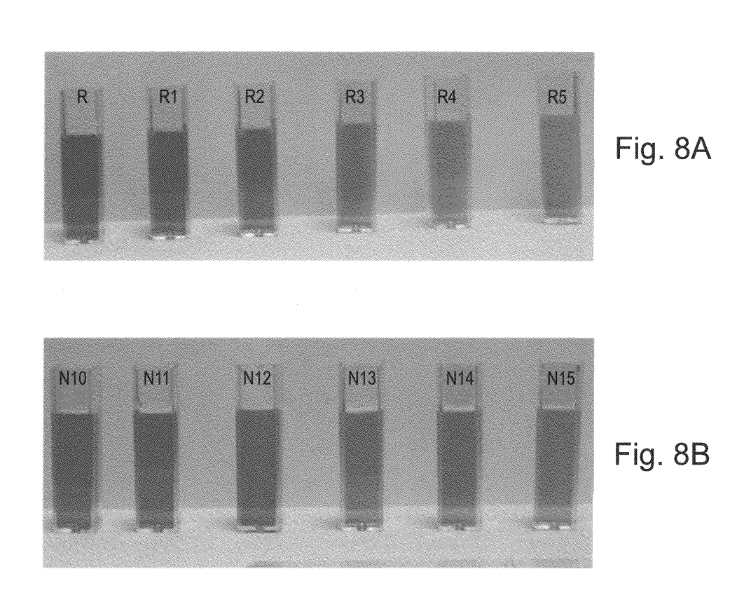

As illustrated in FIGS. 7A-C and 8A-B, the buffering capacity of water comprising nanostructures was higher than the buffering capacity of RO water.

### Example 5

### Buffering Capacity of RF Water

The effect of the RF water on buffering capacity was examined.

Materials and Methods

A few μl drops of Sodium hydroxide 1M were added to raise the pH of 150 ml of RO water (pH=5.8). 50 ml of this water was aliquoted into three bottles.

Three treatments were done:

Bottle 1: no treatment (RO water)

Bottle 2: RO water radiated for 30 minutes with 30 W. The bottle was left to stand on a bench for 10 minutes, before starting the titration (RF water).

Bottle 3: RF water subjected to a second radiation when pH reached 5. After the radiation, the bottle was left to stand on a bench for 10 minutes, before continuing the titration.

Titration was performed by the addition of 1 μl 0.5M Hydrochloric acid to 50 ml water. The titration was finished when the pH value reached below 4.2.

The experiment was performed in triplicates.

Results

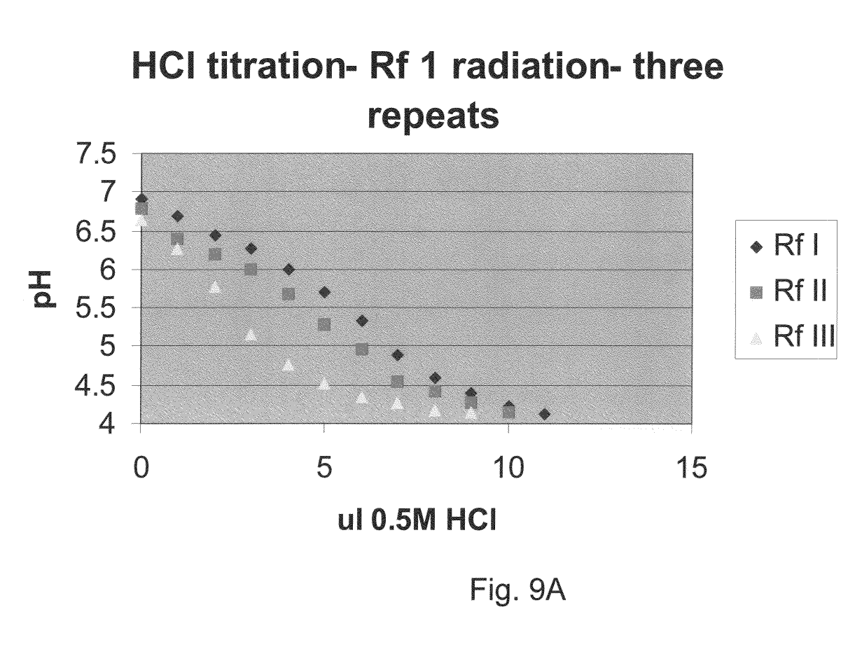

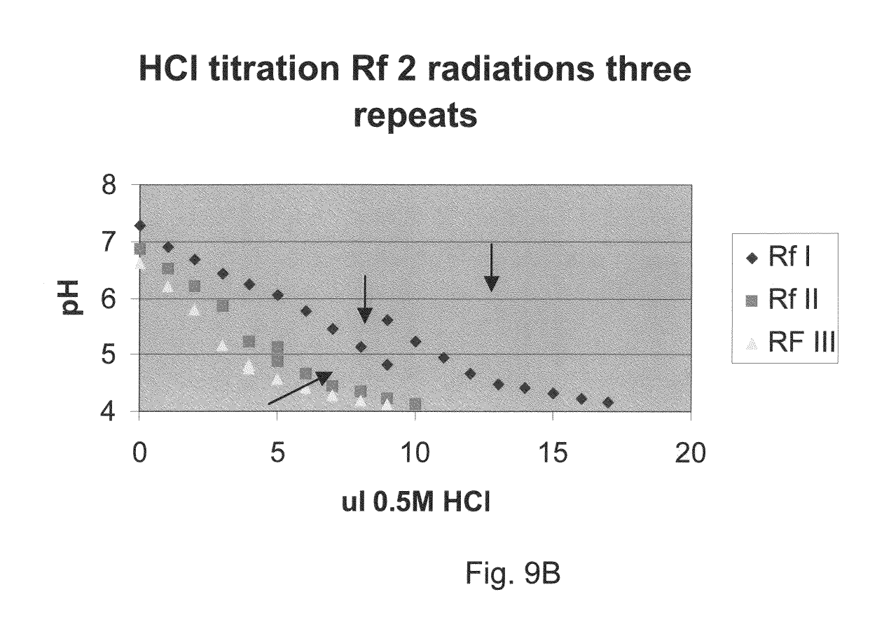

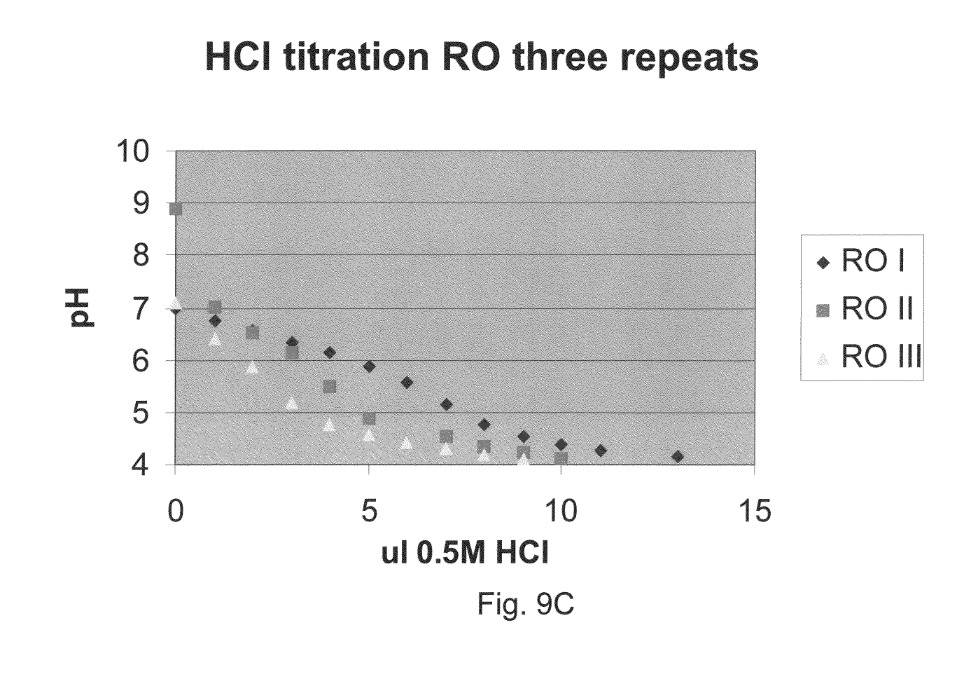

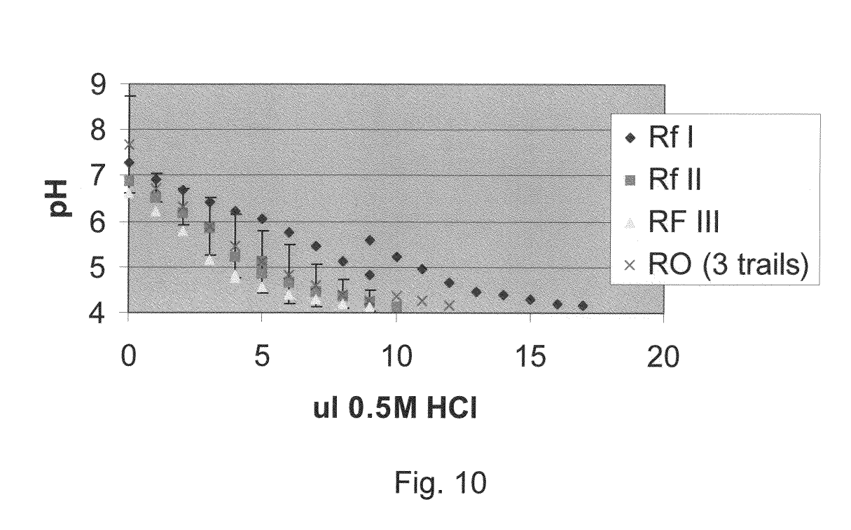

As can be seen from FIGS. 9A-C and FIG. 10, RF water and RF2 water comprise buffering properties similar to those of the carrier composition comprising nanostructures.

### Example 6

### Solvent Capability of the Liquid Composition Comprising Nanostructures

The following experiments were performed in order to ascertain whether the liquid composition comprising nanostructures was capable of dissolving two materials both of which are known not to dissolve in water at a concentration of 1 mg/ml.

A. Dissolving in Ethanol/(Neowater™—Do-Coop Technologies, Israel) Based Solutions

Materials and Methods

Five attempts were made at dissolving the powders in various compositions.

The compositions were as follows:
A. 10 mg powder (red/white)+990 μl Neowater™
B. 10 mg powder (red/white)+990 μl Neowater™ (dehydrated for 90 min).
C. 10 mg powder (red/white)+495 μl Neowater™+495 μl EtOH (50%-50%).
D. 10 mg powder (red/white)+900 μl Neowater™+90 μl EtOH (90%-10%).
E. 10 mg powder (red/white)+820 μl Neowater™+170 μl EtOH (80%-20%).

The tubes were vortexed and heated to 60° C. for 1 hour.

Results
1. The white powder did not dissolve, in all five test tubes.
2. The red powder did dissolve however; it did sediment after a while.
   It appeared as if test tube C dissolved the powder better because the color changed to slightly yellow.

B. Dissolving in Ethanol/(Neowater™—Do-Coop Technologies, Israel) Based Solutions Following Crushing

Materials and Methods

Following crushing, the red powder was dissolved in 4 compositions:

A. ½ mg red powder+49.5 μl RO.
B. ½ mg red powder+49.5 μl Neowater™.
C. ½ mg red powder+9.9 μl EtOH→+39.65 μl Neowater™ (20%-80%).
D. ½ mg red powder+24.75 μl EtOH→24.75 μl Neowater™ (50%-50%).
Total reaction volume: 50 μl.

The tubes were vortexed and heated to 60° C. for 1 hour.

### Results

Following crushing only 20% of ethanol was required in combination with the Neowater™ to dissolve the red powder.

C. Dissolving in Ethanol/(Neowater™—Do-Coop Technologies, Israel) Solutions Following Extensive Crushing

Materials and Methods

Two crushing protocols were performed, the first on the powder alone (vial 1) and the second on the powder dispersed in 100 μl Neowater™ (1%) (vial 2).

The two compositions were placed in two vials on a stirrer to crush the material overnight:

15 hours later, 1000 of Neowater™ was added to 1 mg of the red powder (vial no. 1) by titration of 10 μl every few minutes.

Changes were monitored by taking photographs of the test tubes between 0-24 hours (FIGS. 14F-J).

As a comparison, two tubes were observed one of which comprised the red powder dispersed in 990 μl Neowater™ (dehydrated for 90 min)—1% solution, the other dispersed in a solution comprising 50% ethanol/50% Neowater™)—1% solution. The tubes were heated at 60° C. for 1 hour. The tubes are illustrated in FIGS. 14A-E. Following the 24 hour period, 2 μl from each solution was taken and its absorbance was measured in a nanodrop (FIGS. 15A-C)

Results

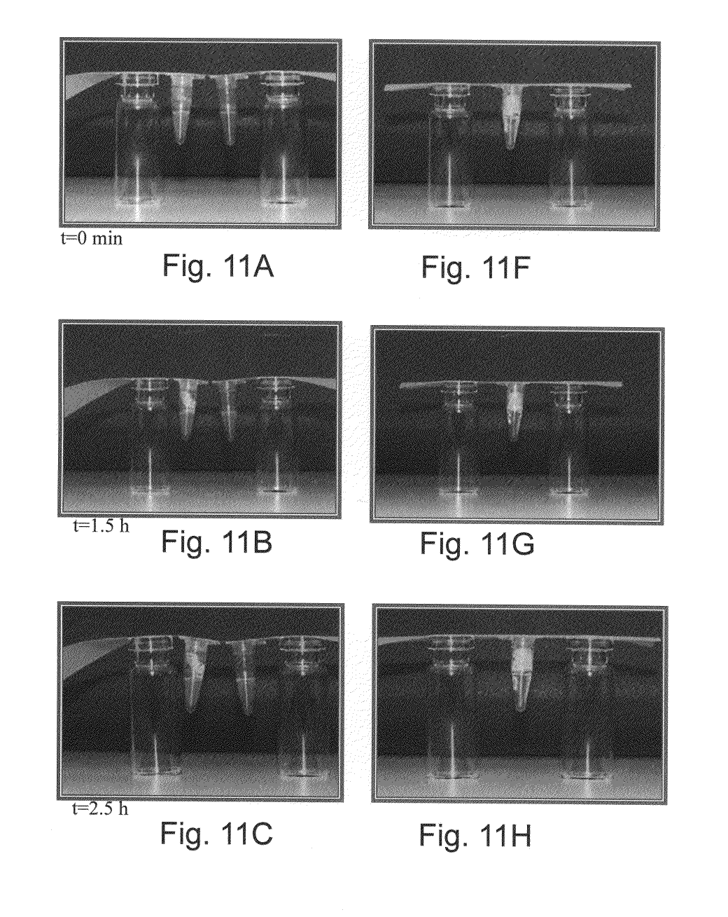

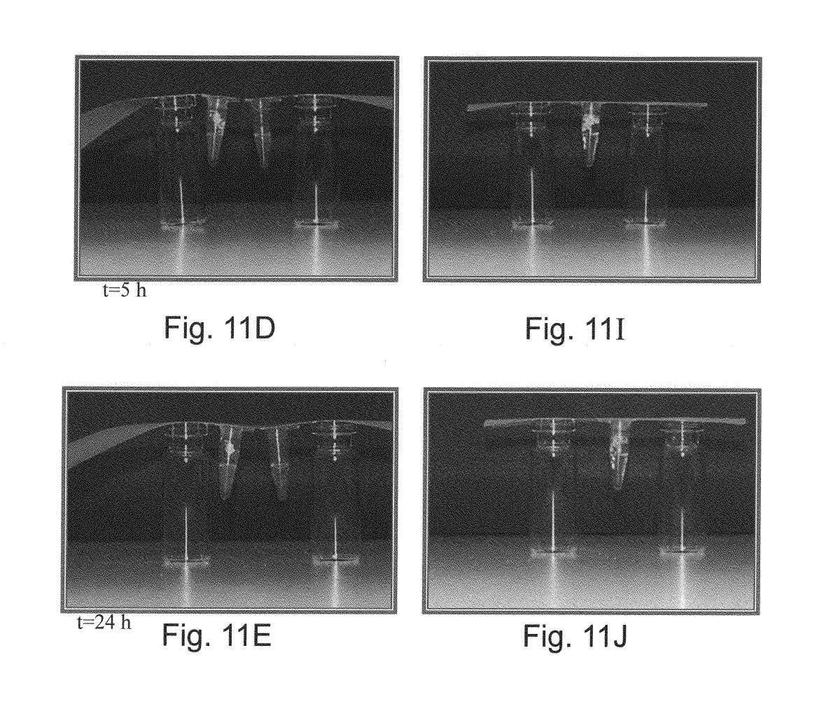

FIGS. 11A-J illustrate that following extensive crushing, it is possible to dissolve the red material, as the material remains stable for 24 hours and does not sink. FIGS. 11A-E however, show the material changing color as time proceeds (not stable).

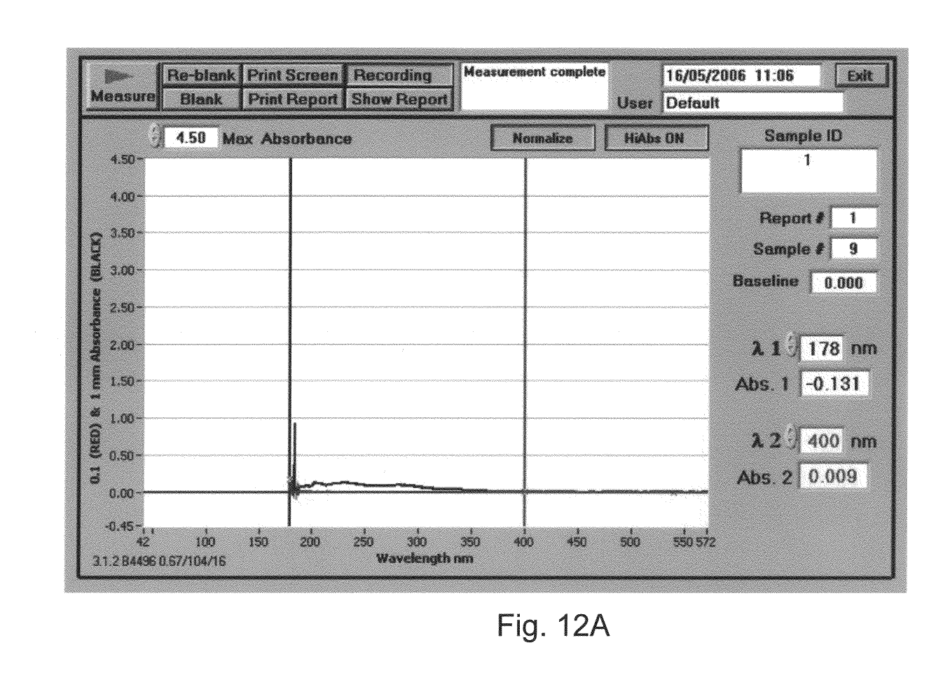

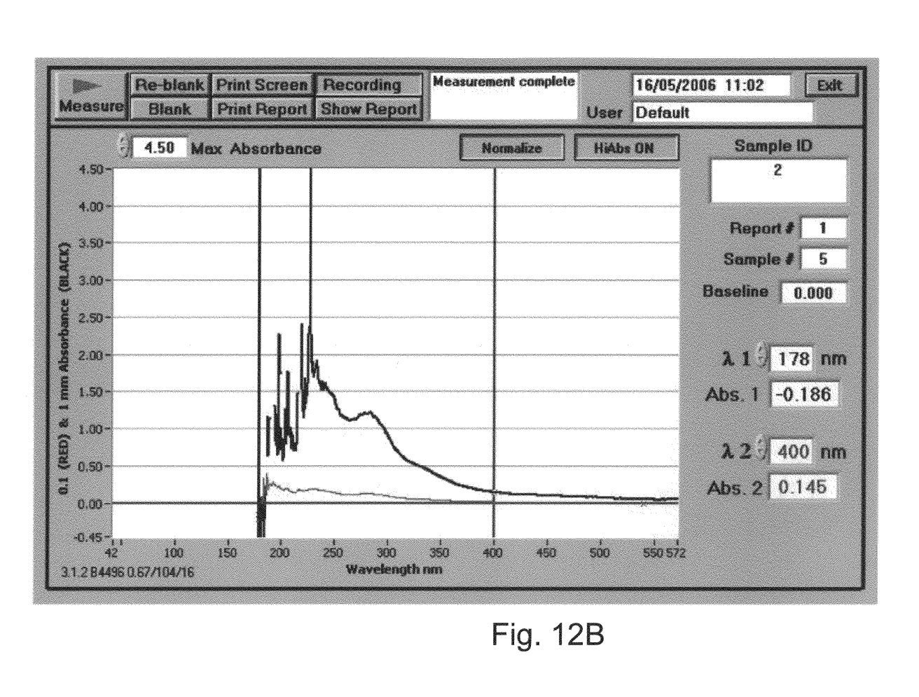

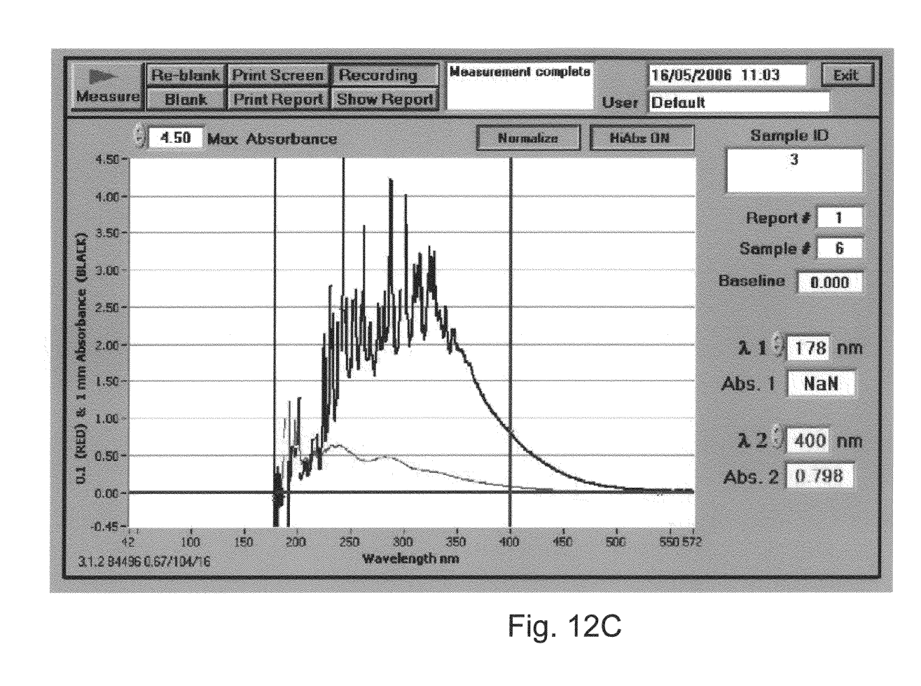

Vial 1 almost didn't absorb (FIG. 12A); solution B absorbance peak was between 220-270 nm (FIG. 12B) with a shift to the left (220 nm) and Solution C absorbance peak was between 250-330 nm (FIG. 12C).

Conclusions

Crushing the red material caused the material to disperse in Neowater™. The dispersion remained over 24 hours. Maintenance of the material in glass vials kept the solution stable 72 h later, both in 100% dehydrated Neowater™ and in EtOH-Neowater™ (50%-50%).

### Example 7

### Capability of the Liquid Composition Comprising Nanostructures to Dissolve Daidzein, Daunrubicine and t-boc Derivative

The following experiments were performed in order to ascertain whether the liquid composition comprising nanostructures was capable of dissolving three materials—Daidzein—daunomycin conjugate (CD-Dau); Daunrubicine (Cerubidine hydrochloride); t-boc derivative of daidzein (tboc-Daid), all of which are known not to dissolve in water.

Materials and Methods

A. Solubilizing CD-Dau—part 1:

Required concentration: 3 mg/ml Neowater.

Properties: The material dissolves in DMSO, acetone, acetonitrile.
Properties: The material dissolves in EtOH.

5 different glass vials were prepared:
1. 5 mg CD-Dau+1.2 ml Neowater™.
2. 1.8 mg CD-Dau+600 μl acetone.
3. 1.8 mg CD-Dau+150 μl acetone+450 μl Neowater™ (25% acetone).
4. 1.8 mg CD-Dau+600 μl 10% *PEG (Polyethylene Glycol).
5. 1.8 mg CD-Dau+600 μl acetone+600 μl Neowater™.
The samples were vortexed and spectrophotometer measurements were performed on vials #1, 4 and 5
The vials were left opened in order to evaporate the acetone (vials #2, 3, and 5).

Results

Vial #1 (100% Neowater): CD-Dau sedimented after a few hours.

Vial #2 (100% acetone): CD-Dau was suspended inside the acetone, although 48 hours later the material sedimented partially because the acetone dissolved the material.

Vial #3 (25% acetone): CD-Dau didn't dissolve very well and the material floated inside the solution (the solution appeared cloudy).

Vial #4 (10% PEG+Neowater): CD-Dau dissolved better than the CD-Dau in vial #1, however it didn't dissolve as well as with a mixture with 100% acetone.

Vial #5: CD-Dau was suspended first inside the acetone and after it dissolved completely Neowater™ was added in order to exchange the acetone. At first acetone dissolved the material in spite of Neowater™ presence. However, as the acetone evaporated the material partially sediment to the bottom of the vial. (The material however remained suspended.

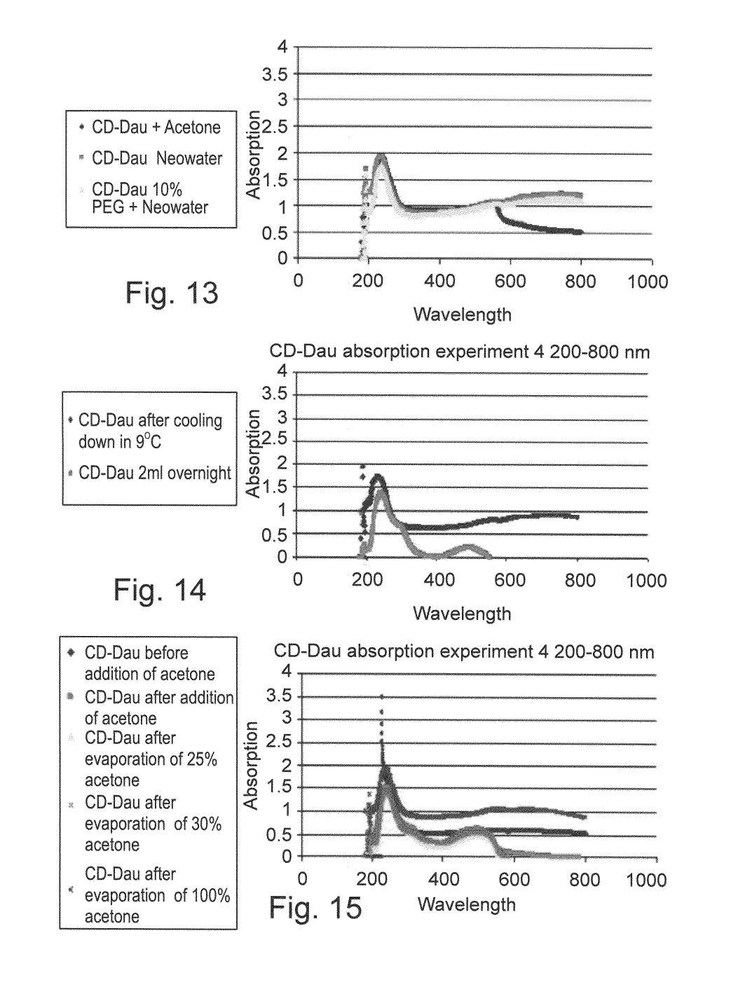

Spectrophotometer measurements (FIG. 13) illustrate the behavior of the material both in the presence and absence of acetone. With acetone there are two peaks in comparison to the material that is suspended with water or with 10% PEG, which in both cases display only one peak.

B. Solubilizing CD-Dau Part 2:

As soon as the acetone was evaporated from solutions #2, 4 and 5, the material sedimented slightly and an additional amount of acetone was added to the vials. This protocol enables the dissolving of the material in the presence of acetone and Neowater™ while at the same time enabling the subsequent evaporation of acetone from the solution (this procedure was performed twice). Following the second cycle the liquid phase was removed from the vile and additional amount of acetone was added to the sediment material. Once the sediment material dissolved it was merged with the liquid phase removed previously. The merged solution was evaporated again. The solution from vial #1 was removed since the material did not dissolve at all and instead 1.2 ml of acetone was added to the sediment to dissolve the material. Later 1.2 ml of 10% PEG+Neowater™ were added also and after some time the acetone was evaporated from the solution. Finalizing these procedures, the vials were merged to one vial (total volume of 3 ml). On top of this final volume 3 ml of acetone were added in order to dissolve the material and to receive a lucid liquefied solution, which was then evaporated again at 50° C. The solution didn't reach equilibrium due to the fact that once reaching such status the solution would have been separated. By avoiding equilibrium, the material hydration status was maintained and kept as liquid. After the solvent evaporated the material was transferred to a clean vial and was closed under vacuum conditions.

C. Solubilizing CD-Dau Part 3:

Another 3 ml of the material (total volume of 6 ml) was generated with the addition of 2 ml of acetone-dissolved material and 1 ml of the remaining material left from the previous experiments.

1.9 ml Neowater™ was added to the vial that contained acetone.

100 μl acetone+100 μl Neowater™ were added to the remaining material. Evaporation was performed on a hot plate adjusted to 50° C. This procedure was repeated 3 times (addition of acetone and its evaporation) until the solution was stable.

The two vials were merged together.

Following the combining of these two solutions, the materials sedimented slightly. Acetone was added and evaporation of the solvent was repeated.

Before mixing the vials (3 ml+2 ml) the first solution prepared in the experiment as described in part 2, hereinabove was incubated at 9° C. over night so as to ensure the solution reached and maintained equilibrium. By doing so, the already dissolved material should not sediment. The following morning the solution's absorption was established and a different graph was obtained (FIG. 14). Following merging of the two vials, absorption measurements were performed again because the material sediment slightly. As a result of the partial sedimentation, the solution was diluted 1:1 by the addition of acetone (5 ml) and subsequently evaporation of the solution was performed at 50° C. on a hot plate. The spectrophotometer read-out of the solution, while performing the evaporation procedure changed due to the presence of acetone (FIG. 15). These experiments imply that when there is a trace of acetone it might affect the absorption readout is received.

B. Solubilizing Daunorubicine (Cerubidine Hydrochloride)

Required concentration: 2 mg/ml

Materials and Methods

2 mg Daunorubicine+1 ml Neowater™ was prepared in one vial and 2 mg of Daunorubicine+1 ml RO was prepared in a second vial.

Results

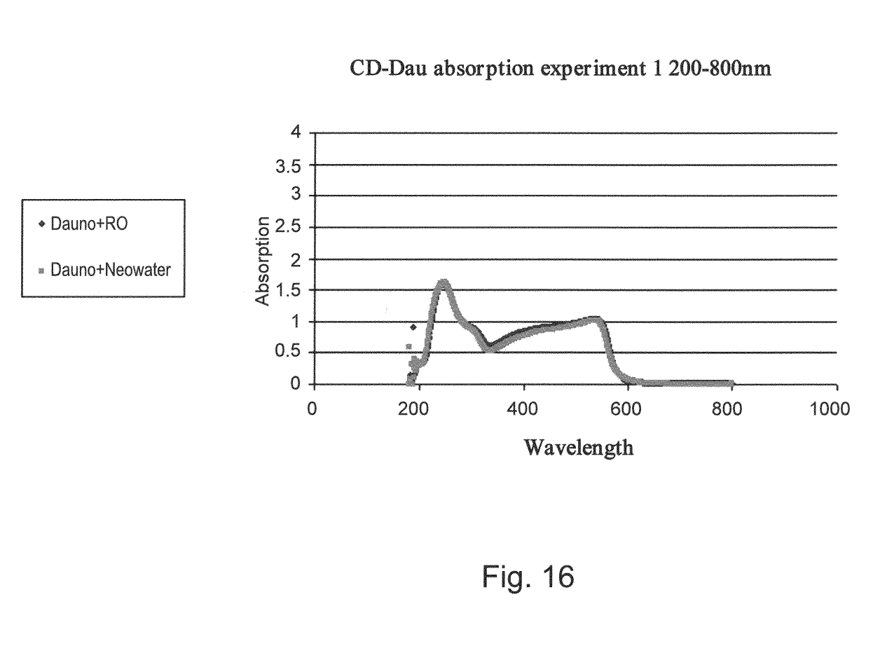

The material dissolved easily both in Neowater™ and RO as illustrated by the spectrophotometer measurements (FIG. 16).

Conclusion

Daunorubicine dissolves without difficulty in Neowater™ and RO.

C. Solubilizing t-boc

Required concentration: 4 mg/ml

### Materials and Methods

1.14 ml of EtOH was added to one glass vial containing 18.5 mg of t-boc (an oily material). This was then divided into two vials and 1.74 ml Neowater™ or RO water was added to the vials such that the solution comprised 25% EtOH. Following spectrophotometer measurements, the solvent was evaporated from the solution and Neowater™ was added to both vials to a final volume of 2.31 ml in each vial. The solutions in the two vials were merged to one clean vial and packaged for shipment under vacuum conditions.

Results

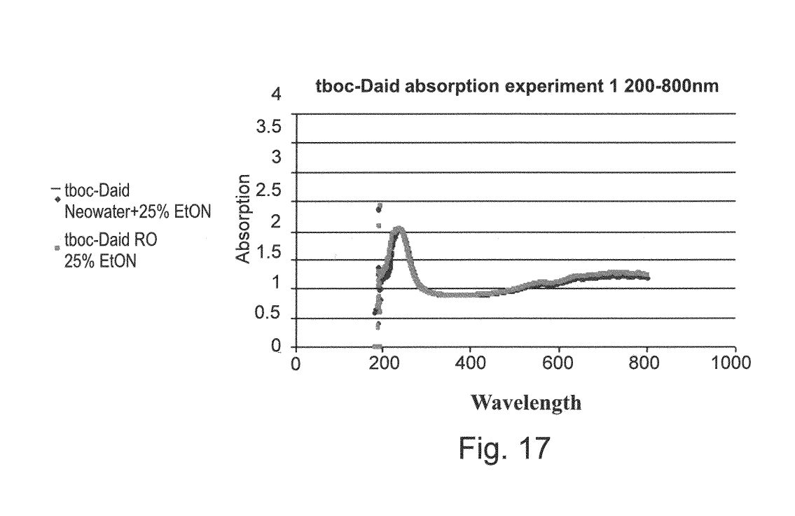

The spectrophotometer measurements are illustrated in FIG. 17. The material dissolved in ethanol. Following addition of Neowater™ and subsequent evaporation of the solvent with heat (50° C.), the material could be dissolved in Neowater™.

Conclusions

The optimal method to dissolve the materials was first to dissolve the material with a solvent (Acetone, Acetic-Acid or Ethanol) followed by the addition of the hydrophilic fluid (Neowater™) and subsequent removal of the solvent by heating the solution and evaporating the solvent.

### Example 8

### Capability of the Liquid Composition Comprising Nanostructures to Dissolve AG-14A and AG-14B

The following experiments were performed in order to ascertain whether the carrier composition comprising nanostructures was capable of dissolving two herbal materials—AG-14A and AG-14B, both of which are known not to dissolve in water at a concentration of 25 mg/ml.

Part 1

Materials and Methods

2.5 mg of each material (AG-14A and AG-14B) was diluted in either Neowater™ alone or a solution comprising 75% Neowater™ and 25% ethanol, such that the final concentration of the powder in each of the four tubes was 2.5 mg/ml. The tubes were vortexed and heated to 50° C. so as to evaporate the ethanol.

Results

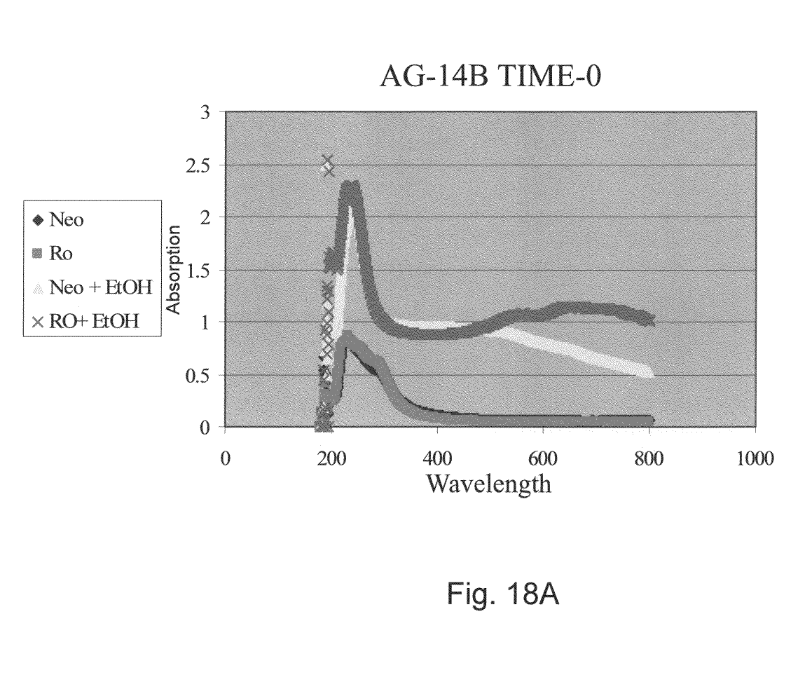

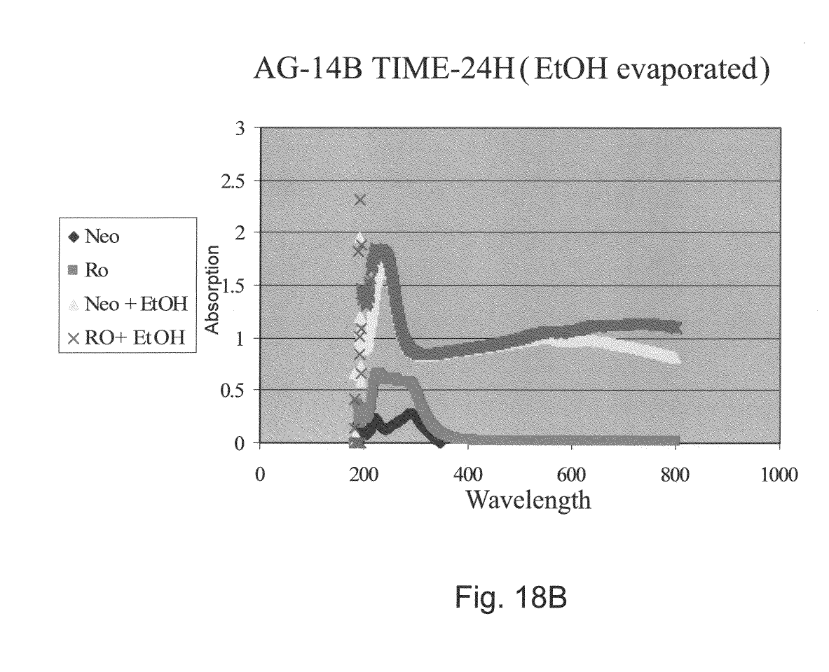

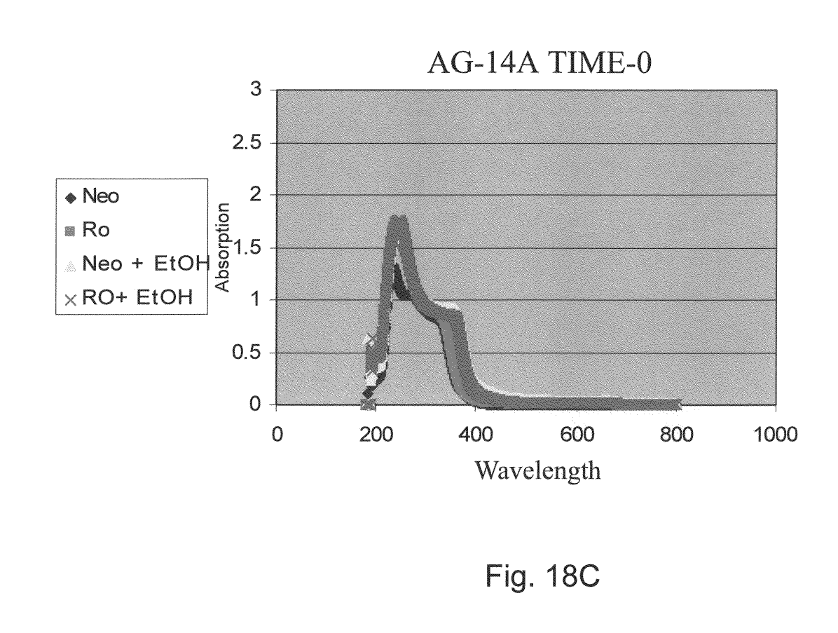

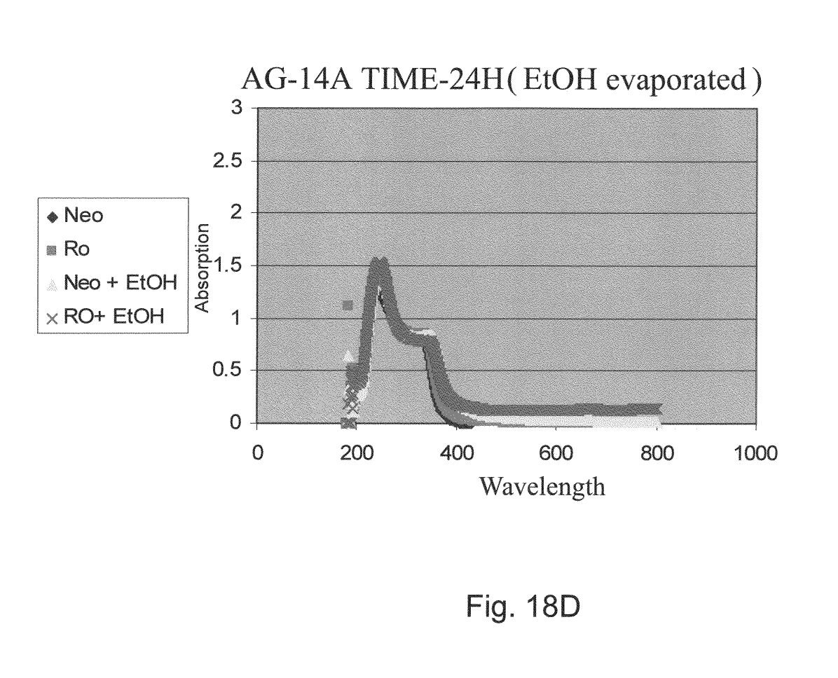

The spectrophotometric measurements of the two herbal materials in Neowater™ in the presence and absence of ethanol are illustrated in FIGS. 18A-D.

Conclusion

Suspension in RO did not dissolve of AG-14B. Suspension of AG-14B in Neowater™ did not aggregate, whereas in RO water, it did.

AG-14A and AG-14B did not dissolve in Neowater/RO.

Part 2

Material and Methods

5 mg of AG-14A and AG-14B were diluted in 62.5 μl EtOH+187.4 μl Neowater™. A further 62.5 μl of Neowater™ were added. The tubes were vortexed and heated to 50° C. so as to evaporate the ethanol.

Results

Suspension in EtOH prior to addition of Neowater™ and then evaporation thereof dissolved AG-14A and AG-14B.

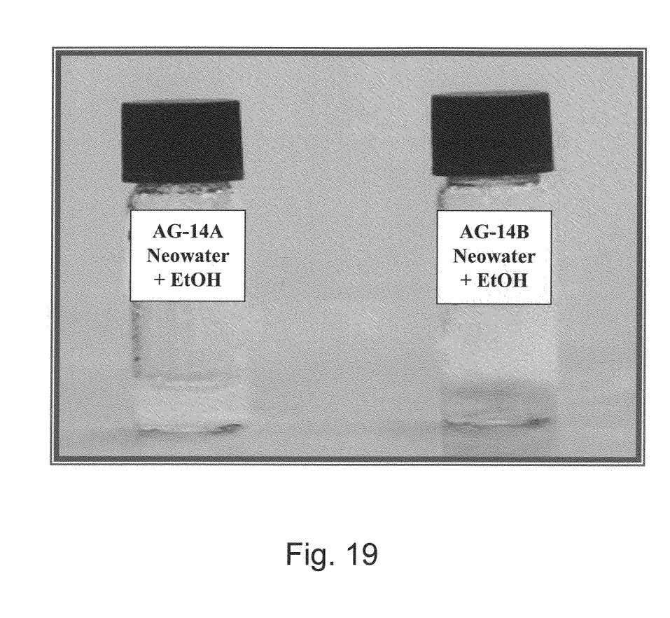

As illustrated in FIG. 19, AG-14A and AG-14B remained stable in suspension for over 48 hours.

### Example 9

### Capability of the Carrier Comprising Nanostructures to Dissolve Peptides

The following experiments were performed in order to ascertain whether the carrier composition comprising nanostructures was capable of dissolving 7 cytotoxic peptides, all of which are known not to dissolve in water. In addition, the effect of the peptides on Skov-3 cells was measured in order to ascertain whether the carrier composition comprising nanostructures influenced the cytotoxic activity of the peptides.

Materials and Methods

Solubilization: All seven peptides (Peptide X, X-5FU, NLS-E, Palm-PFPSYK (CMFU), PFPSYKLRPG-NH₂, NLS-p2-LHRH, and F-LH-RH-palm kGFPSK) were dissolved in Neowater™ at 0.5 mM. Spectrophotometric measurements were taken.

In Vitro Experiment: Skov-3 cells were grown in McCoy's 5A medium, and diluted to a concentration of 1500 cells per well, in a 96 well plate. After 24 hours, 2 μl (0.5 mM, 0.05 mM and 0.005 mM) of the peptide solutions were diluted in 1 ml of McCoy's 5A medium, for final concentrations of 10⁻⁶ M, 10 M and 10⁻⁸ M respectively. 9 repeats were made for each treatment. Each plate contained two peptides in three concentration, and 6 wells of control treatment. 90 μl of McCoy's 5A medium+peptides were added to the cells. After 1 hour, 10 μl of FBS were added (in order to prevent competition). Cells were quantified after 24 and 48 hours in a viability assay based on crystal violet. The dye in this assay stains DNA. Upon solubilization, the amount of dye taken up by the monolayer was quantified in a plate reader.

Results

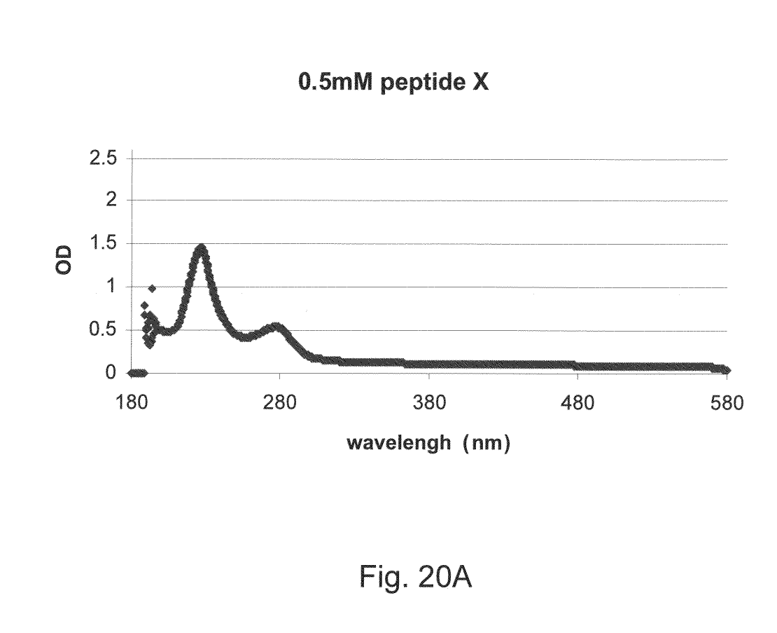

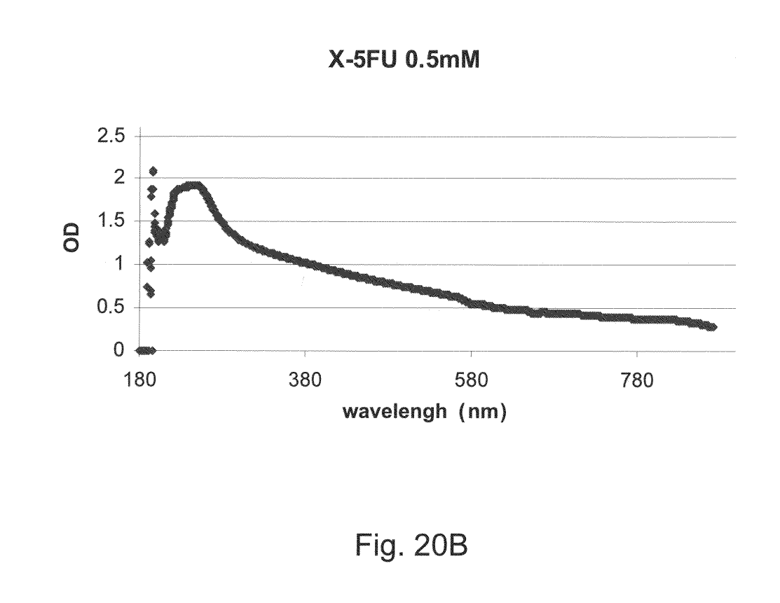

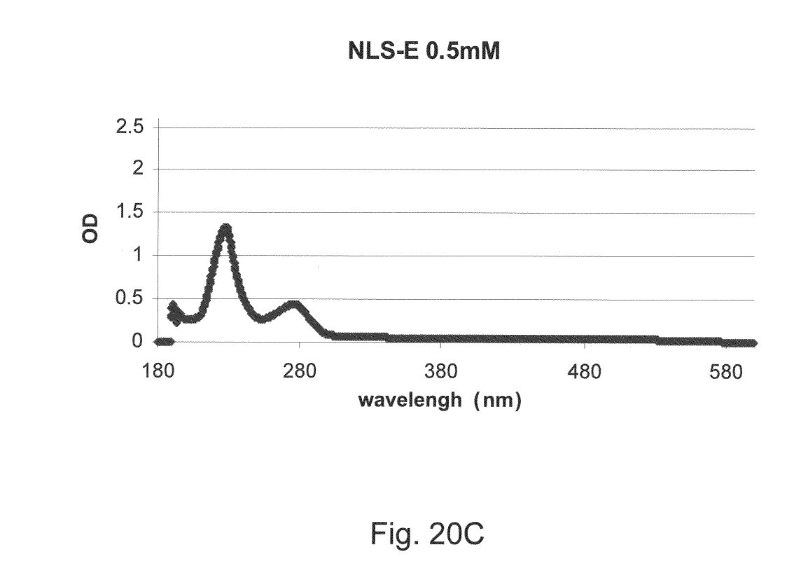

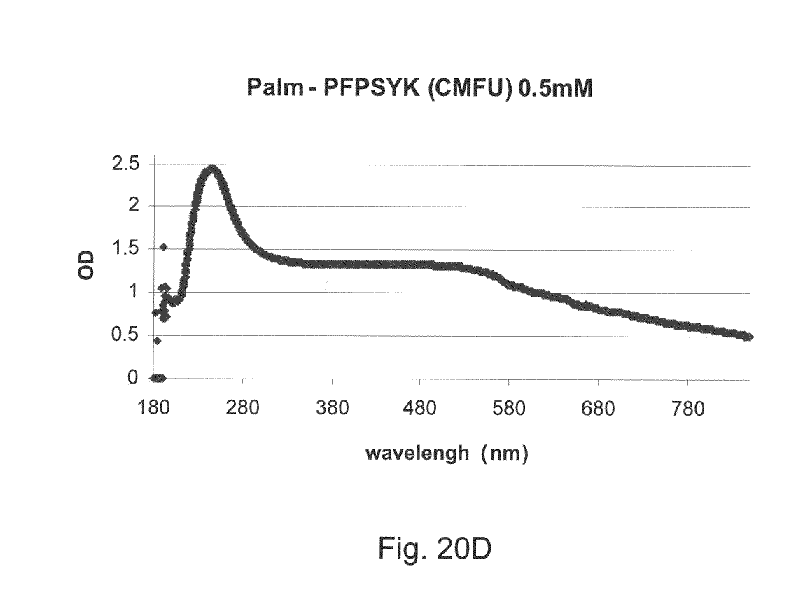

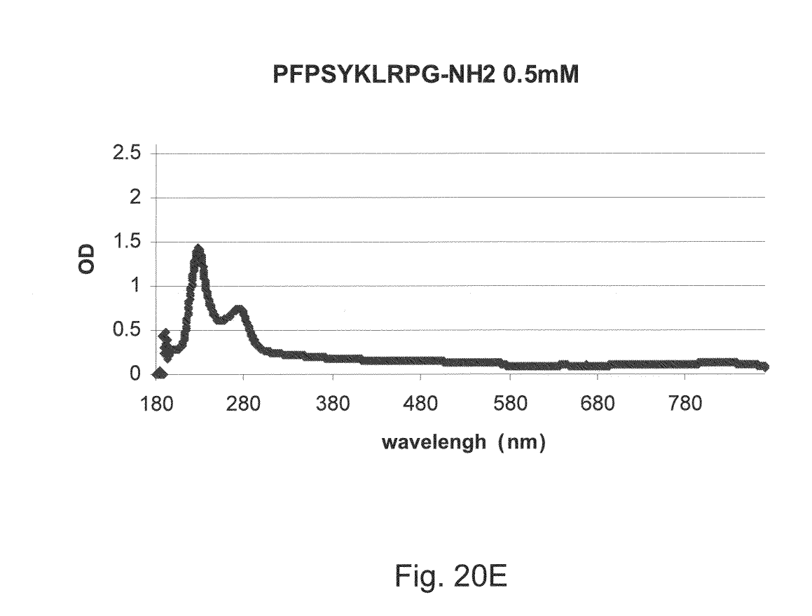

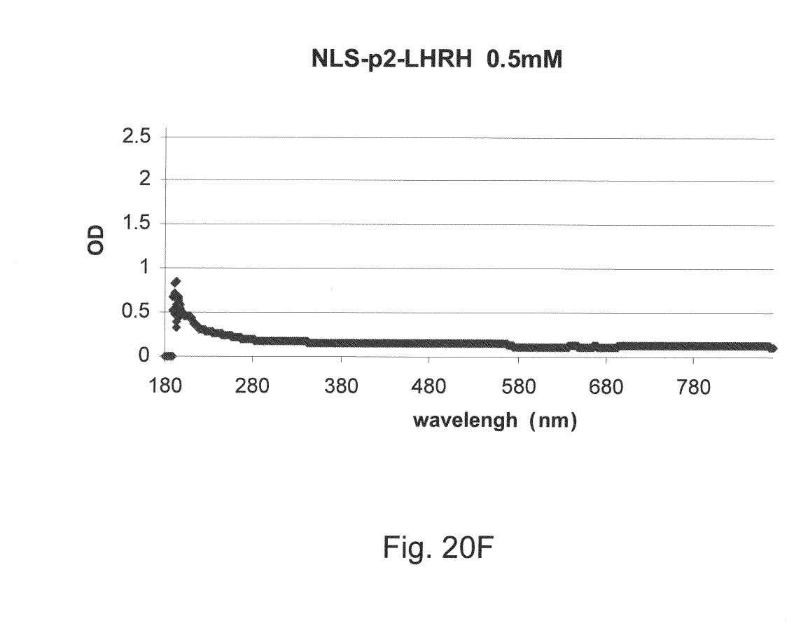

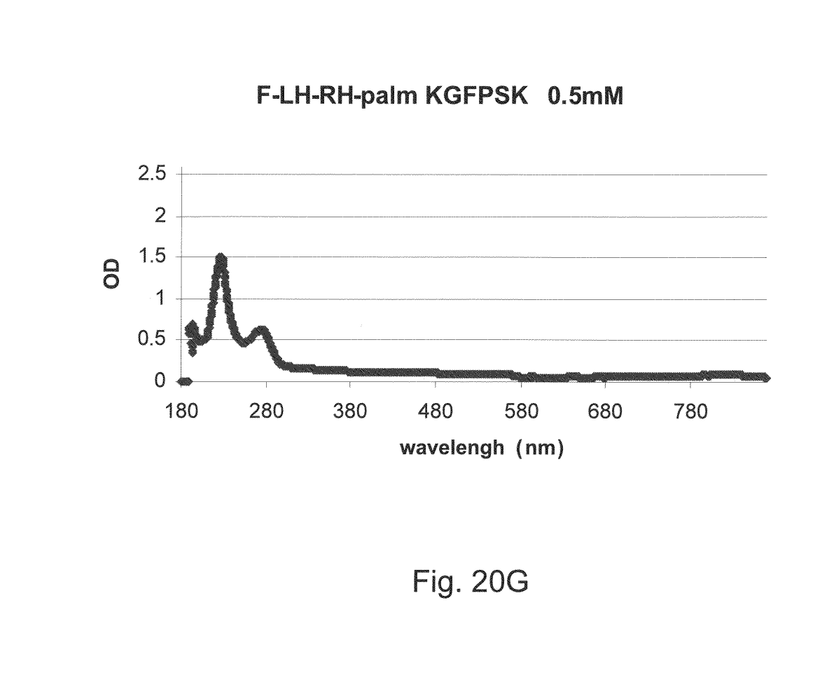

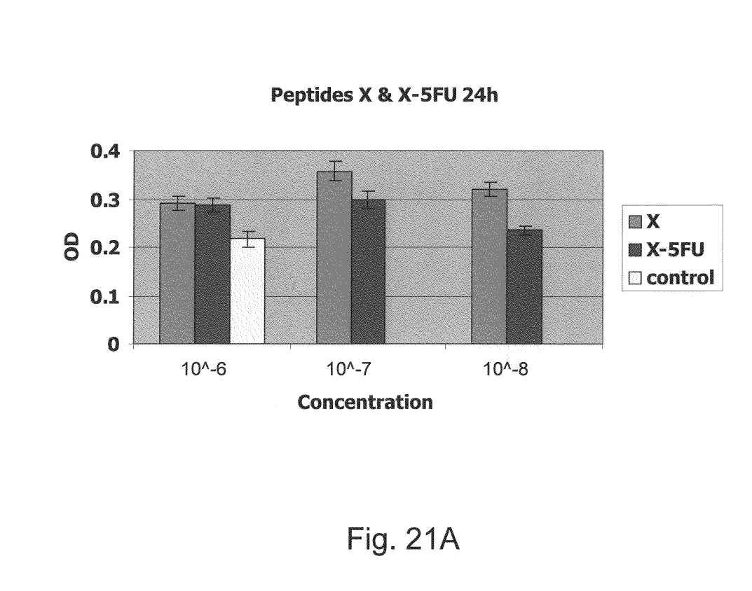

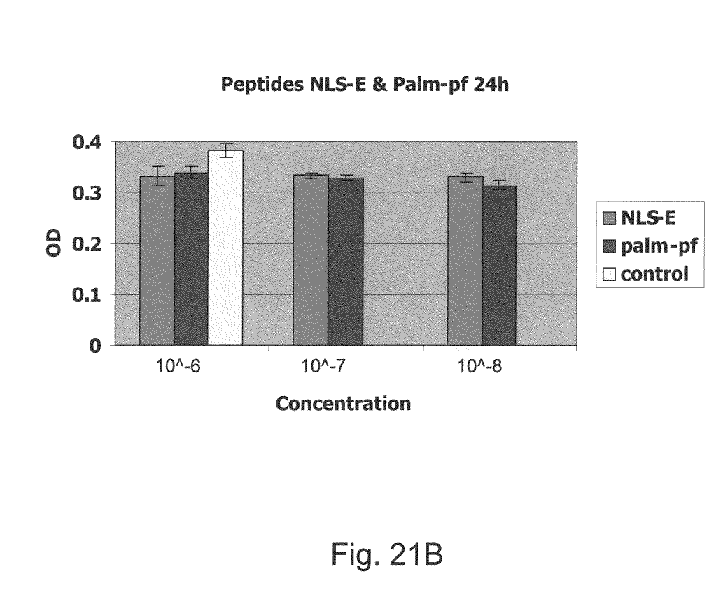

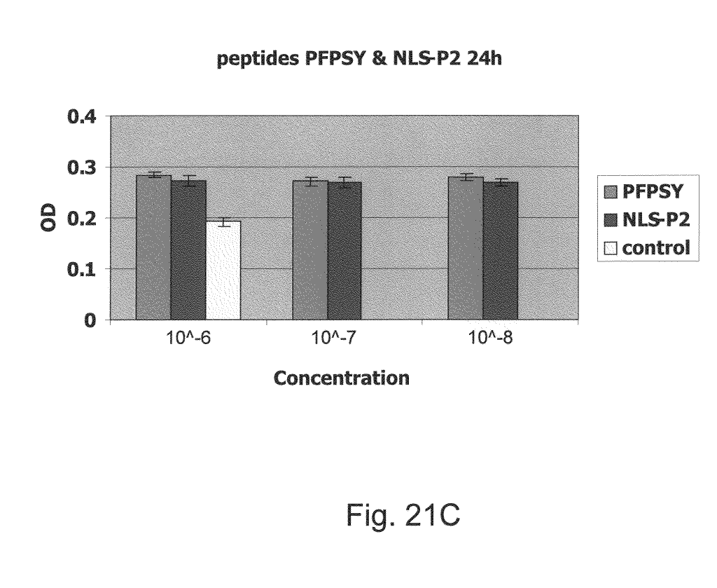

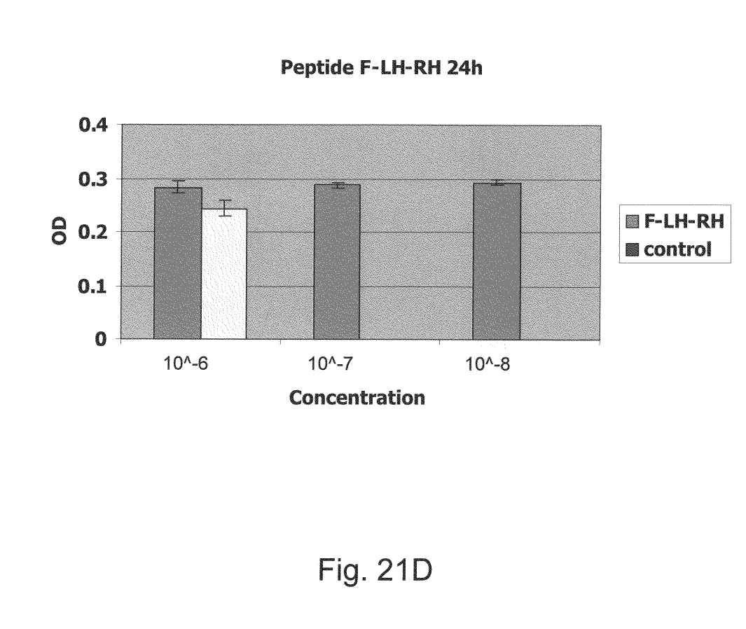

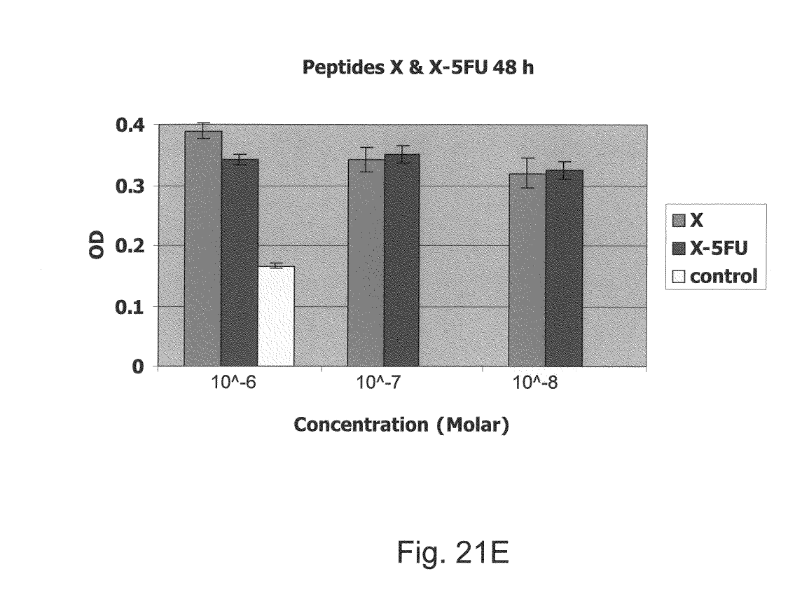

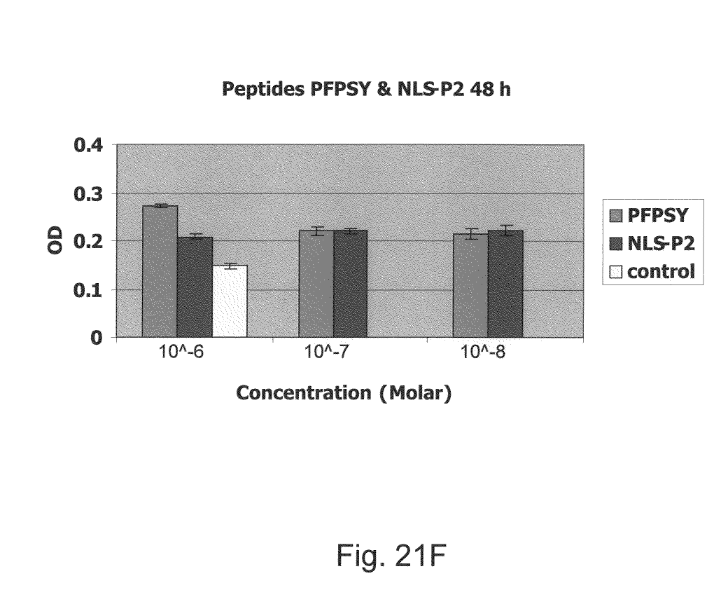

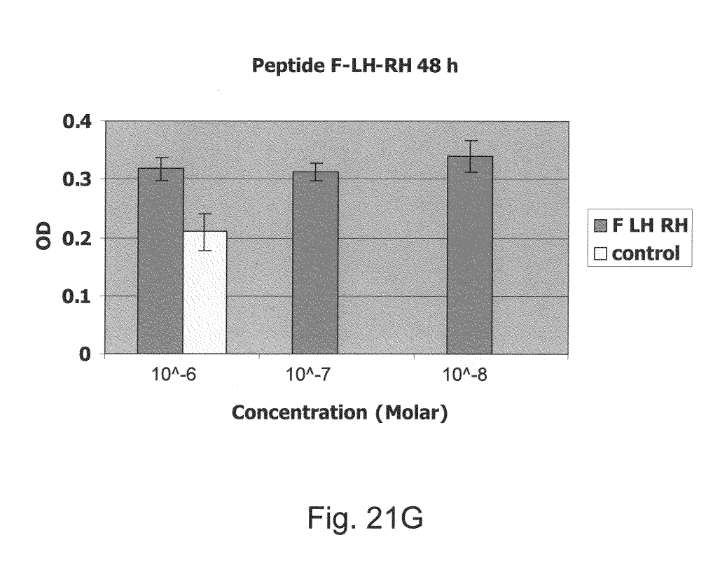

The spectrophotometric measurements of the 7 peptides diluted in Neowater™ are illustrated in FIGS. 20A-G. As illustrated in FIGS. 21A-G, all the dissolved peptides comprised cytotoxic activity.

### Example 10

### Capability of the Liquid Composition Comprising Nanostructures to Dissolve Retinol

The following experiments were performed in order to ascertain whether the liquid composition comprising nanostructures was capable of dissolving retinol.

Materials and Methods

Retinol (vitamin A) was purchased from Sigma (Fluka, 99% HPLC). Retinol was solubilized in Neowater™ under the following conditions.

1% retinol (0.01 gr in 1 ml) in EtOH and Neowater™

0.5% retinol (0.005 gr in 1 ml) in EtOH and Neowater™

0.5% retinol (0.125 gr in 25 ml) in EtOH and Neowater™

0.25% retinol (0.0625 gr in 25 ml) in EtOH and Neowater™. Final EtOH concentration: 1.5%

Absorbance spectrum of retinol in EtOH: Retinol solutions were made in absolute EtOH, with different retinol concentrations, in order to create a calibration graph; absorbance spectrum was detected in a spectrophotometer.

2 solutions with 0.25% and 0.5% retinol in Neowater™ with unknown concentration of EtOH were detected in a spectrophotometer. Actual concentration of retinol is also unknown since some oil drops are not dissolved in the water.

Filtration: 2 solutions of 0.25% retinol in Neowater™ were prepared, with a final EtOH concentration of 1.5%. The solutions were filtrated in 0.44 and 0.2 μl filter.

Results

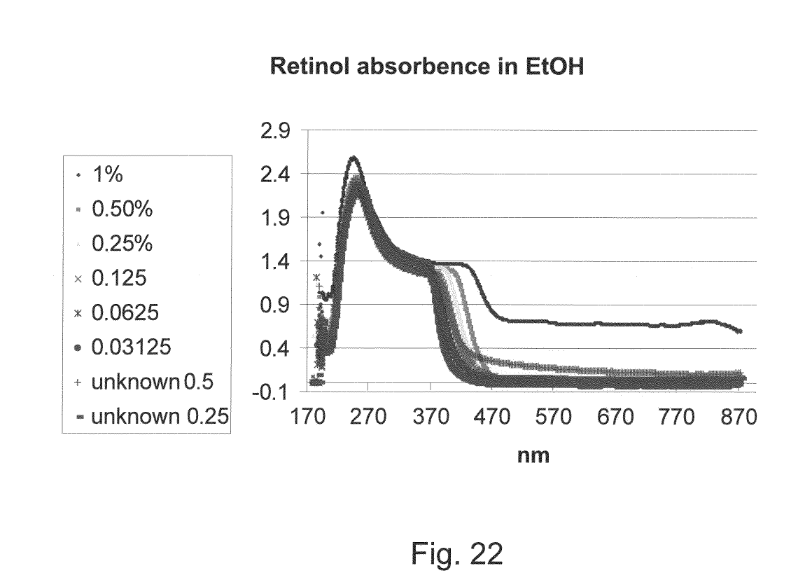

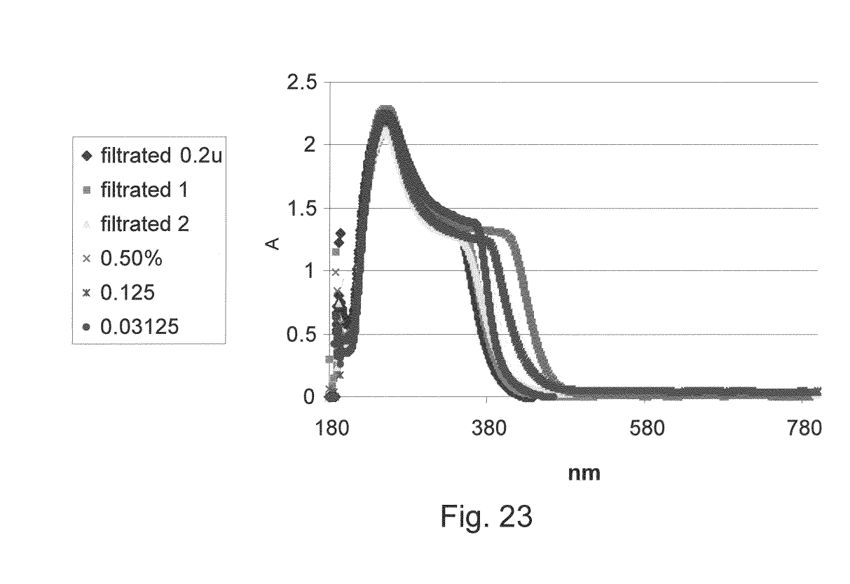

Retinol solubilized easily in alkali Neowater™ rather than acidic Neowater™. The color of the solution was yellow, which faded over time. In the absorbance experiments, 0.5% retinol showed a similar pattern to 0.125% retinol, and 0.25% retinol shows a similar pattern to 0.03125% retinol—see FIG. 22. Since Retinol is unstable in heat; (its melting point is 63° C.), it cannot be autoclaved. Filtration was possible when retinol was fully dissolved (in EtOH). As illustrated in FIG. 23, there is less than 0.03125% retinol in the solutions following filtration. Both filters gave similar results.

### Example 11

### Capability of the Liquid Composition Comprising Nanostructures to Dissolve Material X

The following experiments were performed in order to ascertain whether the liquid composition comprising nanostructures was capable of dissolving material X at a final concentration of 40 mg/ml.

Part 1—solubility in Water and DMSO

Materials and Methods In a first test tube, 25 μl of Neowater™ was added to 1 mg of material “X”. In a second test tube 25 μl of DMSO was added to 1 mg of material “X”. Both test tubes were vortexed and heated to 60° C. and shaken for 1 hour on a shaker.

Results

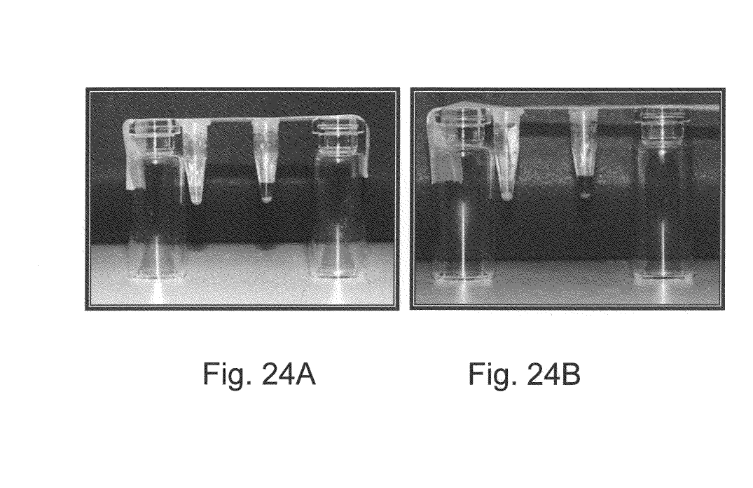

The material did not dissolve at all in Neowater™ (test tube 1). The material dissolved in DMSO and gave a brown-yellow color. The solutions remained for 24-48 hours and their stability was analyzed over time (FIG. 24A-B).

### Conclusions

Neowater™ did not dissolve material “X” and the material sedimented, whereas DMSO almost completely dissolved material “X”.

Part 2—Reduction of DMSO and Examination of the Material Stability/Kinetics in Different Solvents Over the Course of Time.

Materials and Methods

6 different test tubes were analyzed each containing a total reaction volume of 25 μl:

1. 1 mg “X”+25 μl Neowater™ (100%).

2. 1 mg “X”+12.5 μl DMSO→12.5 μl Neowater™ (50%).

3. 1 mg “X”+12.5 μl DMSO+12.5 μl Neowater™ (50%).

4. 1 mg “X”+6.25 μl DMSO+18.75 μl Neowater™ (25%).

5. 1 mg “X”+25 μl Neowater™+sucrose* (10%).

6. 1 mg+12.5 μl DMSO+12.5 μl dehydrated Neowater™** (50%).

* 0.1 g sucrose+1 ml (Neowater™)=10% Neowater™+sucrose
** Dehydrated Neowater™ was achieved by dehydration of Neowater™ for 90 min at 60° C.

All test tubes were vortexed, heated to 60° C. and shaken for 1 hour.

Results

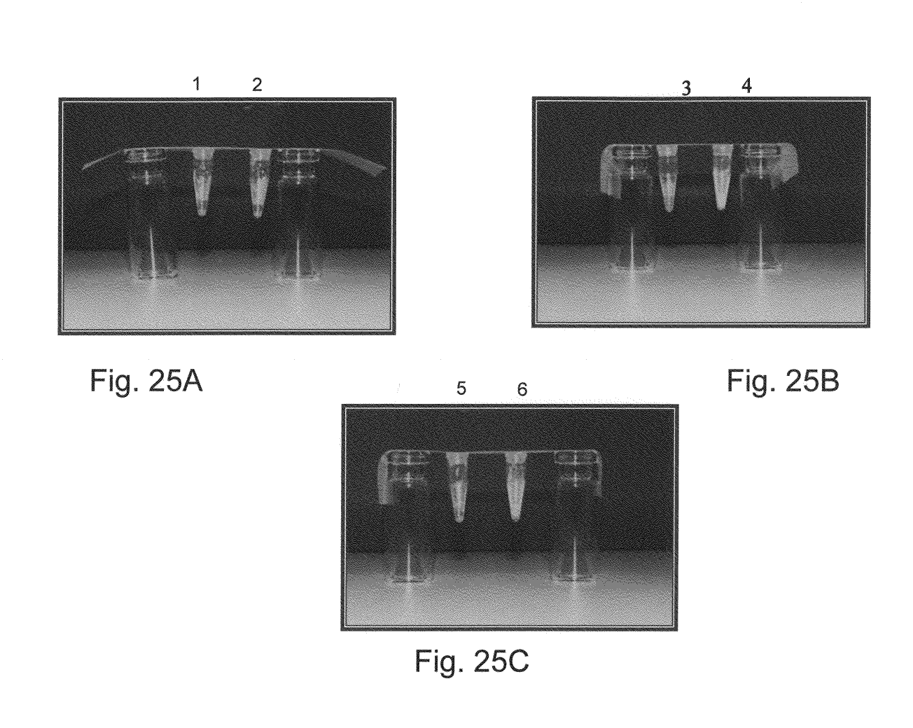

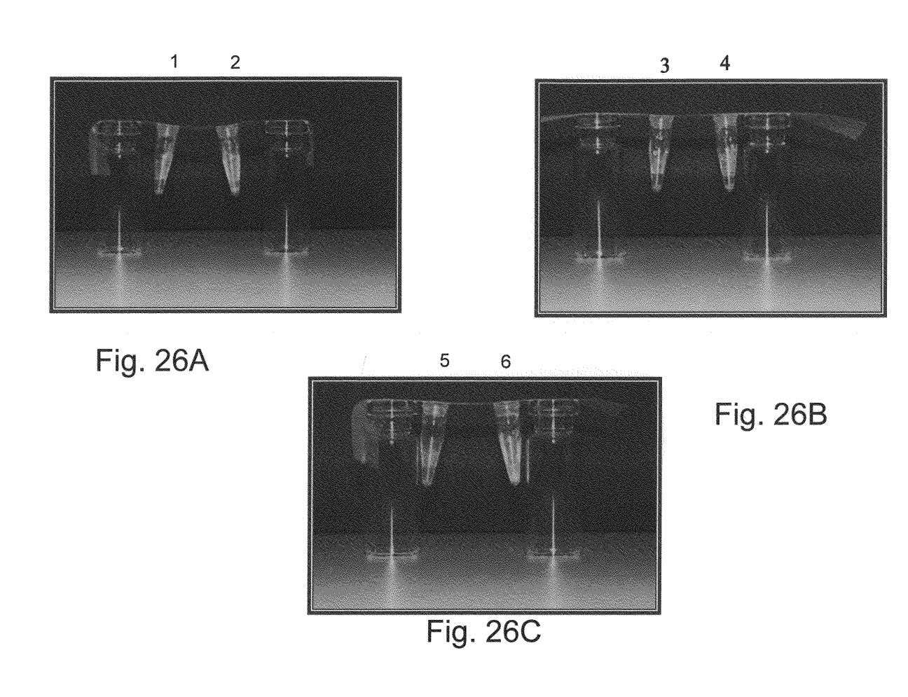

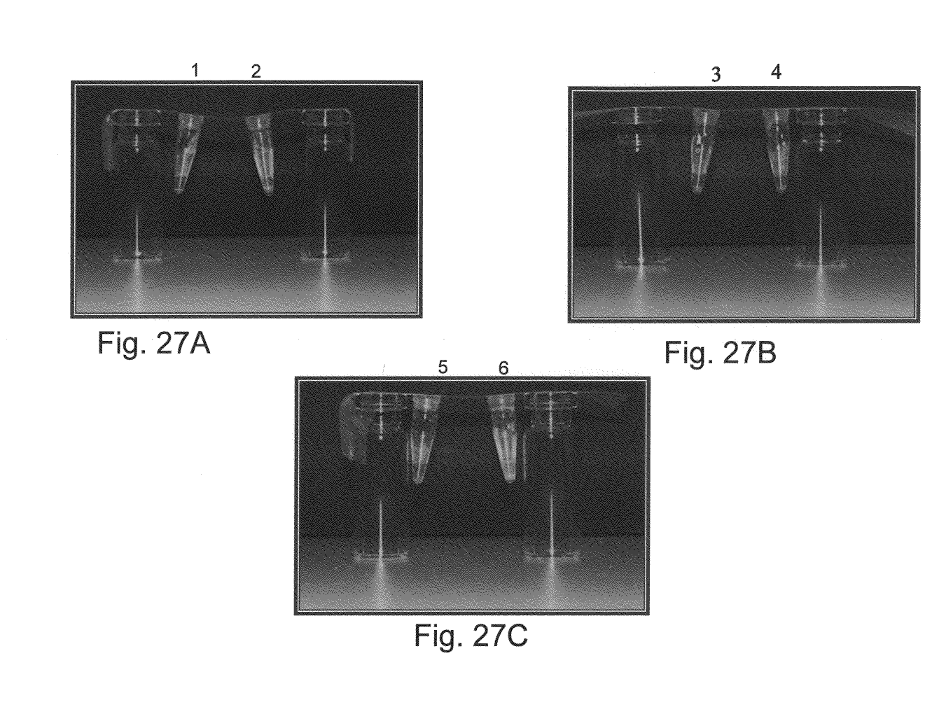

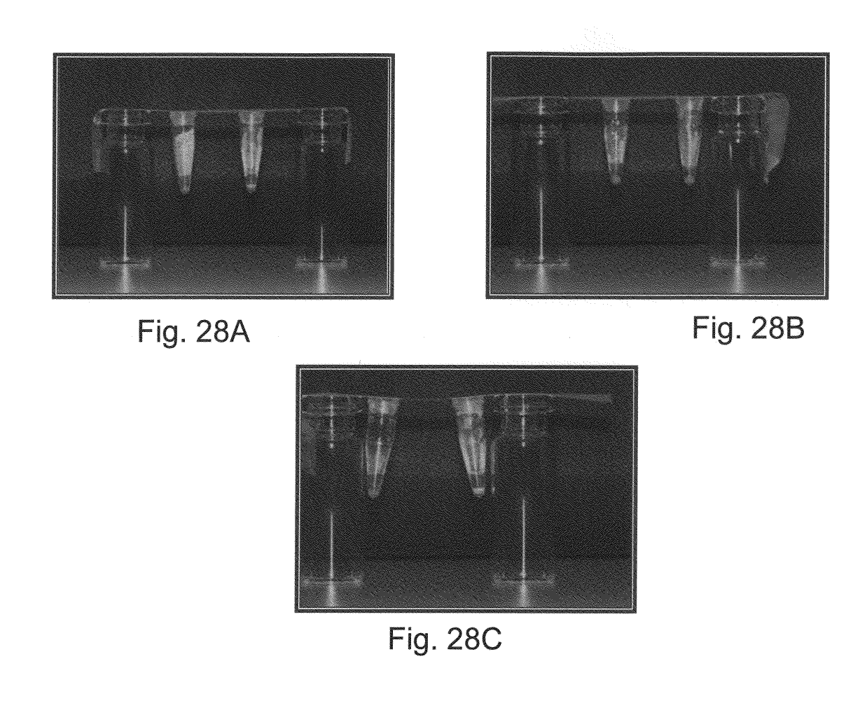

The test tubes comprising the 6 solvents and substance X at time 0 are illustrated in FIGS. 25A-C. The test tubes comprising the 6 solvents and substance X at 60 minutes following solubilization are illustrated in FIGS. 26A-C. The test tubes comprising the 6 solvents and substance X at 120 minutes following solubilization are illustrated in FIGS. 27A-C. The test tubes comprising the 6 solvents and substance X 24 hours following solubilization are illustrated in FIGS. 28A-C.

Conclusion

Material “X” did not remain stable throughout the course of time, since in all the test tubes the material sedimented after 24 hours.

There is a different between the solvent of test tube 2 and test tube 6 even though it contains the same percent of solvents. This is because test tube 6 contains dehydrated Neowater™ which is more hydrophobic than non-dehydrated Neowater™.

Part 3 Further Reduction of DMSO and Examination of the Material Stability/Kinetics in Different Solvents Over the Course of Time.

Materials and Methods

1 mg of material “X”+50 μl DMSO were placed in a glass tube. 50 μl of Neowater™ were titred (every few seconds 5 μl) into the tube, and then 500 μl of a solution of Neowater™ (9% DMSO+91% Neowater™) was added.

In a second glass tube, 1 mg of material “X”+50 μl DMSO were added. 50 μl of RO were titred (every few seconds 5 μl) into the tube, and then 500 μl of a solution of RO (9% DMSO+91% RO) was added.

Results

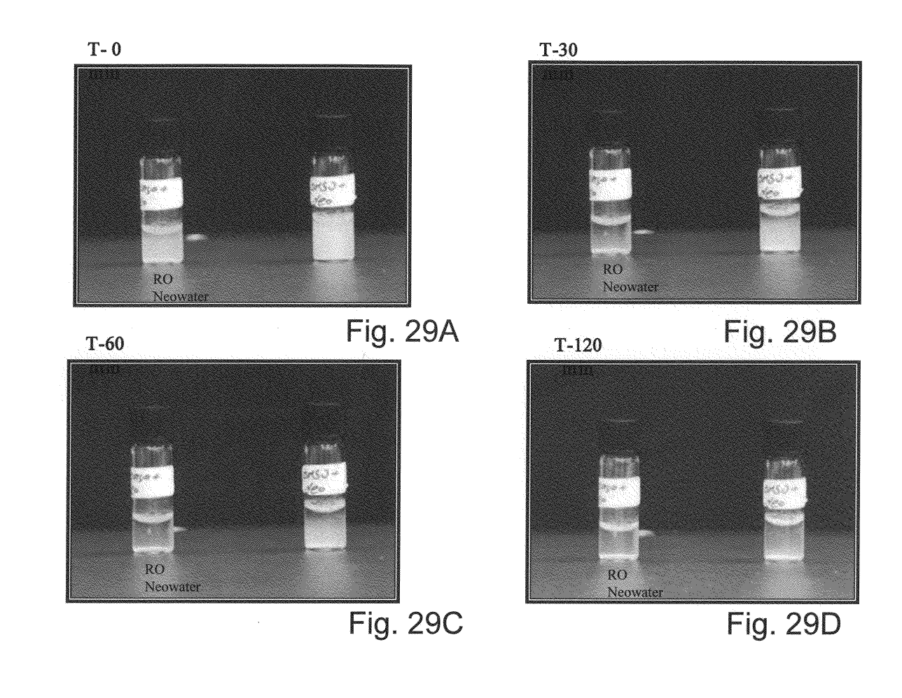

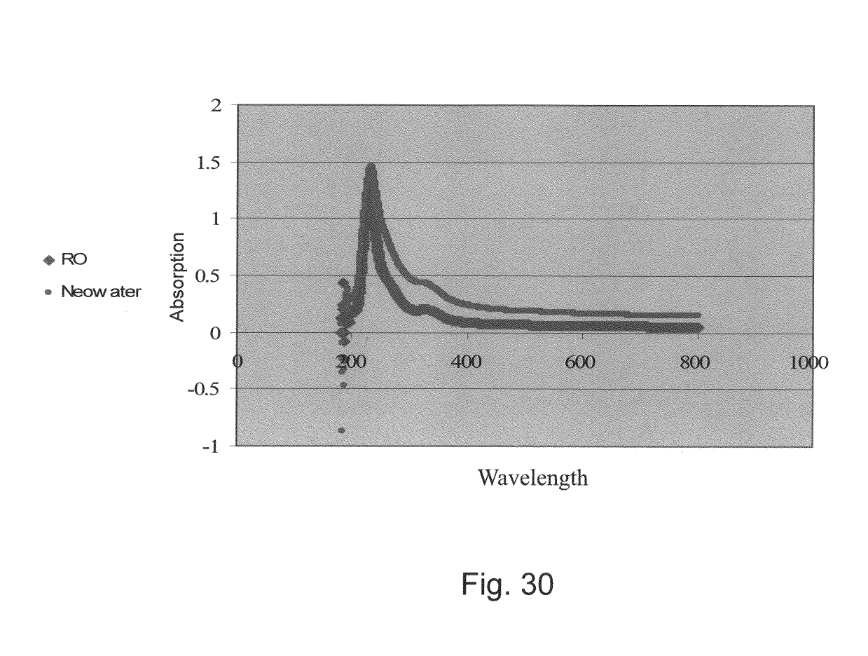

As illustrated in FIGS. 29A-D, material “X” remained dispersed in the solution comprising Neowater™, but sedimented to the bottom of the tube, in the solution comprising RO water. FIG. 30 illustrates the absorption characteristics of the material dispersed in RO/Neowater™ and acetone 6 hours following vortexing.

Conclusion

It is clear that material “X” dissolves differently in RO compare to Neowater™, and it is more stable in Neowater™ compare to RO. From the spectrophotometer measurements (FIG. 30), it is apparent that the material “X” dissolved better in Neowater™ even after 5 hours, since, the area under the graph is larger than in RO. It is clear the Neowater™ hydrates material “X”. The amount of DMSO may be decreased by 20-80% and a solution based on Neowater™ may be achieved that hydrates material “X” and disperses it in the Neowater™.

### Example 12

### Capability of the Liquid Composition Comprising Nanostructures to Dissolve SPL 2101 and SPL 5217

The following experiments were performed in order to ascertain whether the liquid composition comprising nanostructures was capable of dissolving material SPL 2101 and SPL 5217 at a final concentration of 30 mg/ml.

Materials and Methods

SPL 2101 was dissolved in its optimal solvent (ethanol)—FIG. 31A and SPL 5217 was dissolved in its optimal solvent (acetone)—FIG. 31B. The two compounds were put in glass vials and kept in dark and cool environment. Evaporation of the solvent was performed in a dessicator and over a long period of time Neowater™ was added to the solution until there was no trace of the solvents.

Results

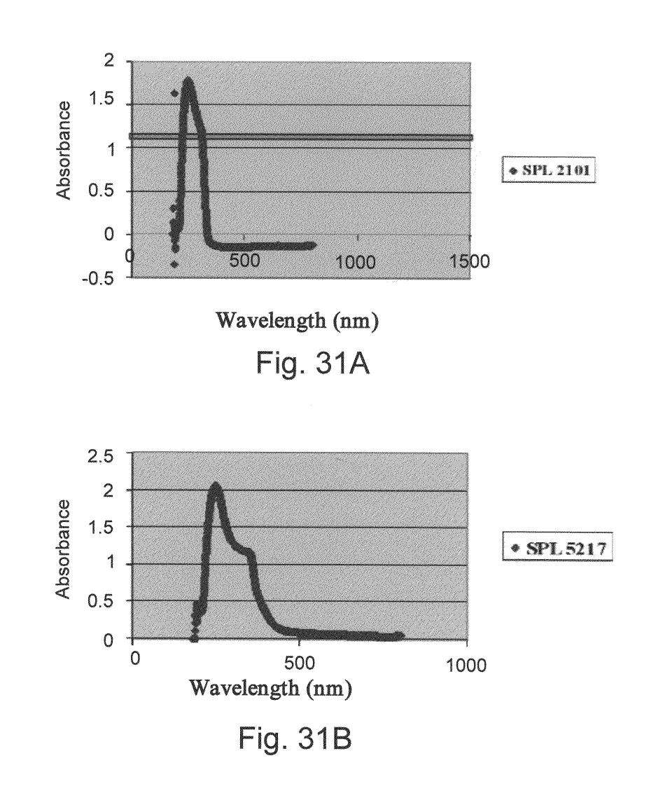

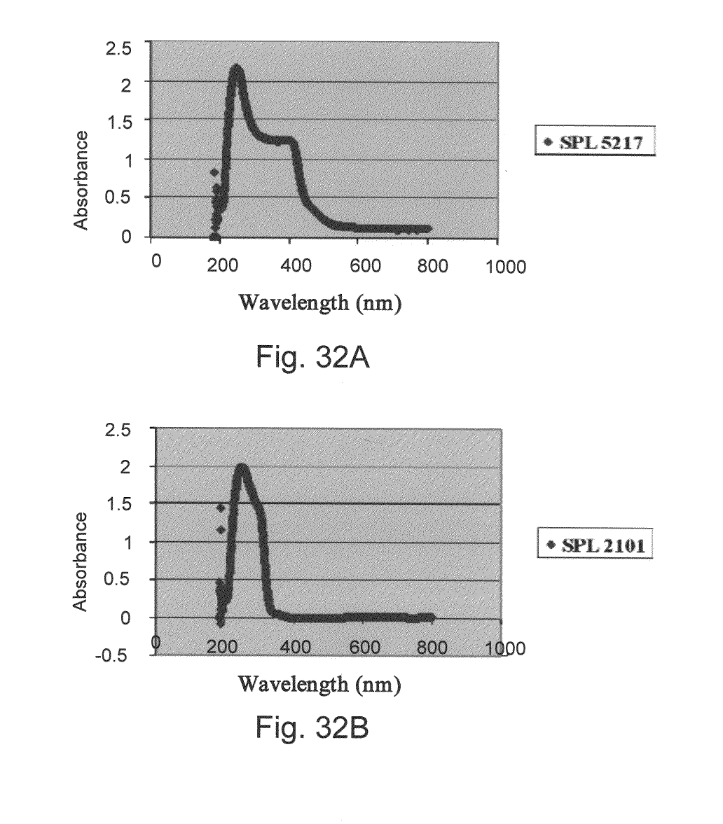

SPL 2101 & SPL 5217 dissolved in Neowater™ as illustrated by the spectrophotometer data in FIGS. 32A-B.

### Example 13

### Capability of the Liquid Composition Comprising Nanostructures to Dissolve Taxol

The following experiments were performed in order to ascertain whether the carrier composition comprising nanostructures was capable of dissolving material taxol (Paclitaxel) at a final concentration of 0.5 mM.

Materials and Methods

Solubilization: 0.5 mM Taxol solution was prepared (0.0017 gr in 4 ml) in either DMSO or Neowater™ with 17% EtOH. Absorbance was detected with a spectrophotometer.

Cell viability assay: 150,000 293T cells were seeded in a 6 well plate with 3 ml of DMEM medium. Each treatment was grown in DMEM medium based on RO or Neowater™. Taxol (dissolved in Neowater™ or DMSO) was added to final concentration of 1.666 μM (10 μl of 0.5 mM Taxol in 3 ml medium). The cells were harvested following a 24 hour treatment with taxol and counted using trypan blue solution to detect dead cells.

Results

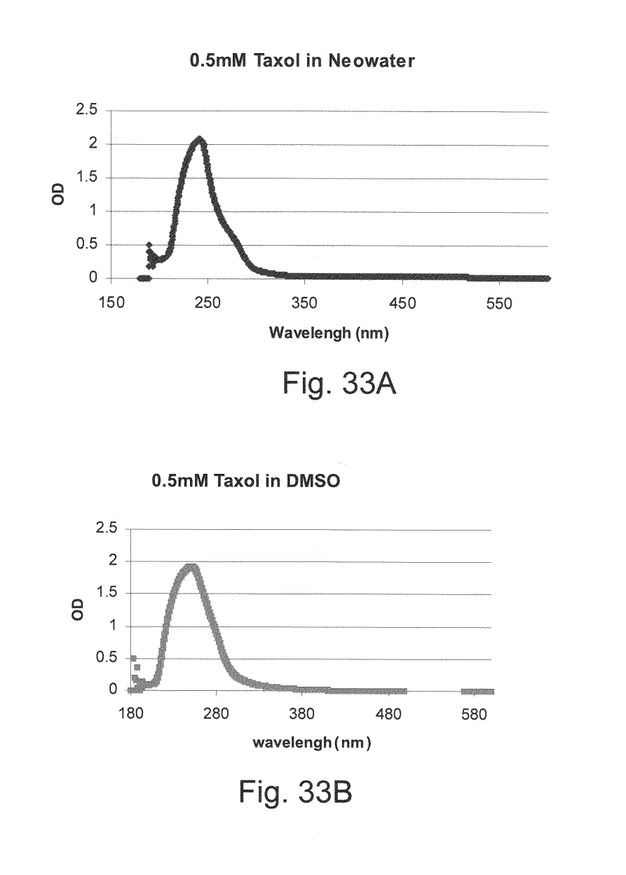

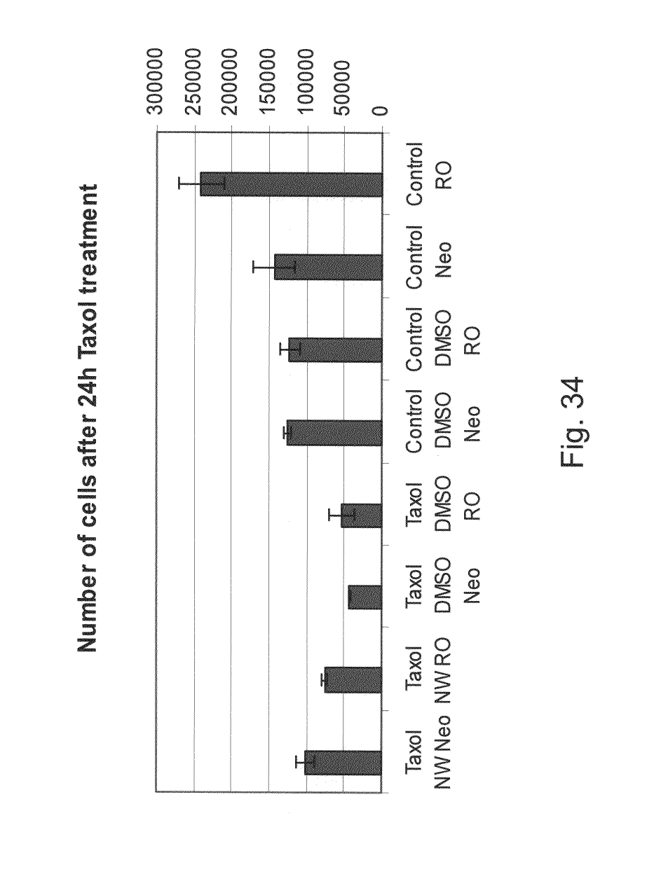

Taxol dissolved both in DMSO and Neowater™ as illustrated in FIGS. 33A-B. The viability of the 293T cells following various solutions of taxol is illustrated in FIG. 34.

Conclusion

Taxol comprised a cytotoxic effect following solution in Neowater™.

### Example 14

### Stabilizing Effect of the Liquid Composition Comprising Nanostructures

The following experiment was performed to ascertain if the liquid composition comprising nanostructures effected the stability of a protein.

Materials and Methods

Two commercial Taq polymerase enzymes (Peq-lab and Bio-lab) were checked in a PCR reaction to determine their activities in ddH₂O(RO) and carrier comprising nanostructures (Neowater™—Do-Coop technologies, Israel). The enzyme was heated to 95° C. for different periods of time, from one hour to 2.5 hours. 2 types of reactions were made:

Water only—only the enzyme and water were boiled.

All inside—all the reaction components were boiled: enzyme, water, buffer, dNTPs, genomic DNA and primers.

Following boiling, any additional reaction component that was required was added to PCR tubes and an ordinary PCR program was set with 30 cycles.

Results

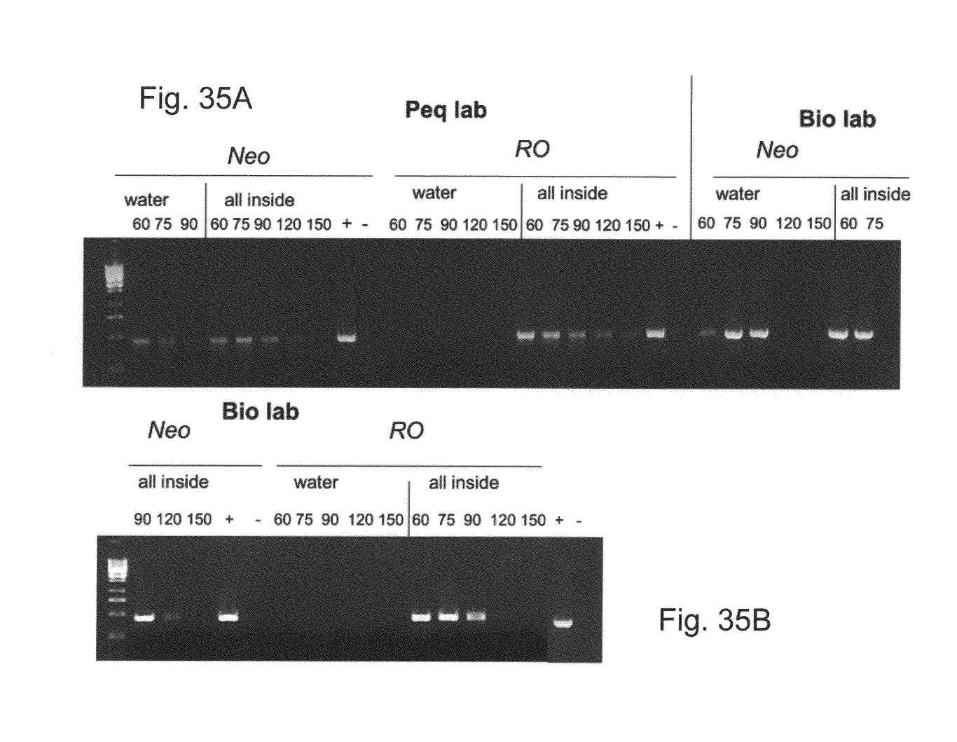

As illustrated in FIGS. 35A-B, the carrier composition comprising nanostructures protected the enzyme from heating, both under conditions where all the components were subjected to heat stress and where only the enzyme was subjected to heat stress. In contrast, RO water only protected the enzyme from heating under conditions where all the components were subjected to heat stress.

### Example 15

### Further Illustration of the Stabilizing Effect of the Carrier Comprising Nanostructures

The following experiment was performed to ascertain if the carrier composition comprising nanostructures effected the stability of two commercial Taq polymerase enzymes (Peq-lab and Bio-lab).

Materials and Methods

The PCR reactions were set up as follows:

Peq-lab samples: 20.4 μl of either the carrier composition comprising nanostructures (Neowater™—Do-Coop technologies, Israel) or distilled water (Reverse Osmosis=RO).

0.1 μl Taq polymerase (Peq-lab, Taq DNA polymerase, 5 U/μl)

Three samples were set up and placed in a PCR machine at a constant temperature of 95° C. Incubation time was: 60, 75 and 90 minutes.

Following boiling of the Taq enzyme the following components were added:

2.5 μl 10× reaction buffer Y (Peq-lab)
0.5 μl dNTPs 10 mM (Bio-lab)
1 μl primer GAPDH mix 10 μmol/μl
0.5 μl genomic DNA 35 μg/μl

Biolab Samples

18.9 μl of either carrier comprising nanostructures (Neowater™—Do-Coop technologies, Israel) or distilled water (Reverse Osmosis=RO).

0.1 μl Taq polymerase (Bio-lab, Taq polymerase, 5 U/Five samples were set up and placed in a PCR machine at a constant temperature of 95° C. Incubation time was: 60, 75, 90 120 and 150 minutes.

Following boiling of the Taq enzyme the following components were added:

2.5 μl TAQ 10× buffer Mg-free (Bio-lab)

### 1.5 μl MgCl₂ 25 mM (Bio-lab)

0.5 μl dNTPs 10 mM (Bio-lab)
1 μl primer GAPDH mix (10 μmol/0.5 μl genomic DNA (35 μg/μl)

For each treatment (Neowater or RO) a positive and negative control were made. Positive control was without boiling the enzyme. Negative control was without boiling the enzyme and without DNA in the reaction. A PCR mix was made for the boiled taq assays as well for the control reactions.

Samples were placed in a PCR machine, and run as follows:

PCR Program:

1. 94° C. 2 minutes denaturation

2. 94° C. 30 seconds denaturation

3. 60° C. 30 seconds annealing

4. 72° C. 30 seconds elongation

repeat steps 2-4 for 30 times

5. 72° C. 10 minutes elongation

Results

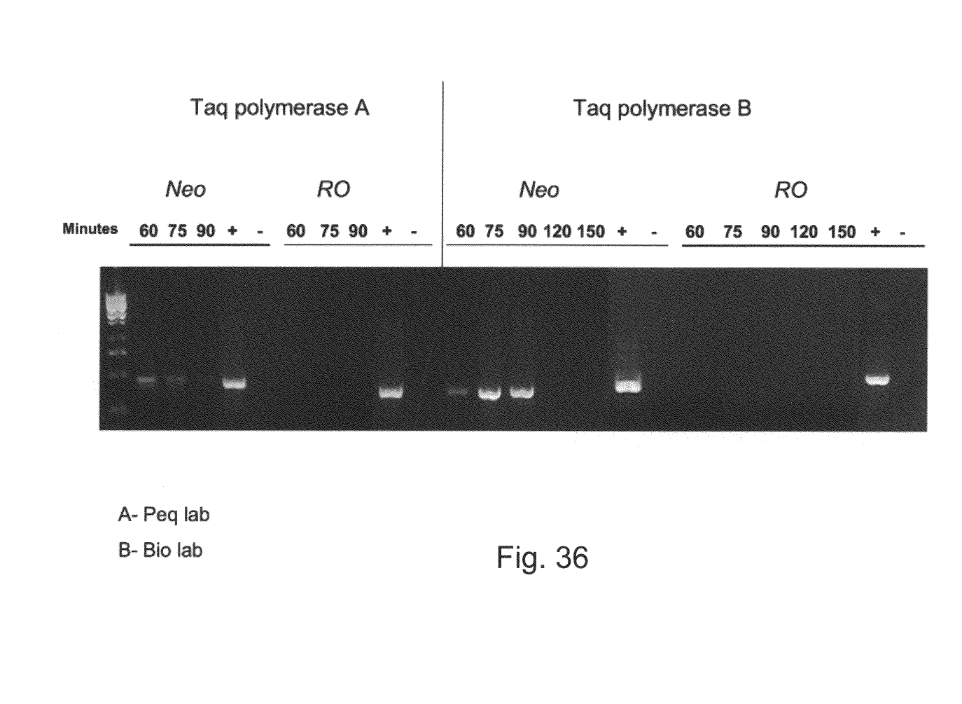

As illustrated in FIG. 36, the liquid composition comprising nanostructures protected both the enzymes from heat stress for up to 1.5 hours.

### Example 16

### Further Evidence that the Liquid Composition Comprising Nanostructures is Capable of Dissolving Taxol

The following experiments were performed in order to ascertain whether the carrier composition comprising nanostructures was capable of dissolving material taxol (Paclitaxel) at a final concentration of 0.5 mM in the presence of 0.08% ethanol.

Materials and Methods

Solubilization: 0.5 mM Taxol solution was prepared (0.0017 gr in 4 ml). Taxol was dissolved in ethanol and exchanged to Neowater™ using an RT slow solvent exchange procedure which extended for 20 days. At the end of the procedure, less than 40% ethanol remained in the solution, leading to 0.08% of ethanol in the final administered concentration. The solution was sterilized using a 0.2 μm filter. Taxol was separately prepared in DMSO (0.5 mM). Both solutions were kept at −20° C. Absorbance was detected with a spectrophotometer.

Cell viability assay: 2000 PC3 cells were seeded per well of a 96-well plate with 100 μl of RPMI based medium with 10% FCS. 24 hours post seeding, 2 μl, 1 μl and 0.5 μl of 0.5 mM taxol were diluted in 1 ml of RPMI medium, reaching a final concentration of 1 μM, 0.5 μM and 0.25 μM respectively. A minimum number of eight replicates were run per treatment. Cell proliferation was assessed by quantifying the cell density using a crystal violet colorimetric assay 24 hours after the addition of taxol.

24 hours post treatment, the cells were washed with PBS and fixed with 4% paraformaldehyde. Crystal violet was added and incubated at room temperature for 10 minutes. After washing the cells three times, a solution with 100 M Sodium Citrate in 50% ethanol was used to elute the color from the cells. Changes in optical density were read at 570 nm using a spectrophotometric plate reader. Cell viability was expressed as a percentage of the control optical density, deemed as 100%, after subtraction of the blank.

Results

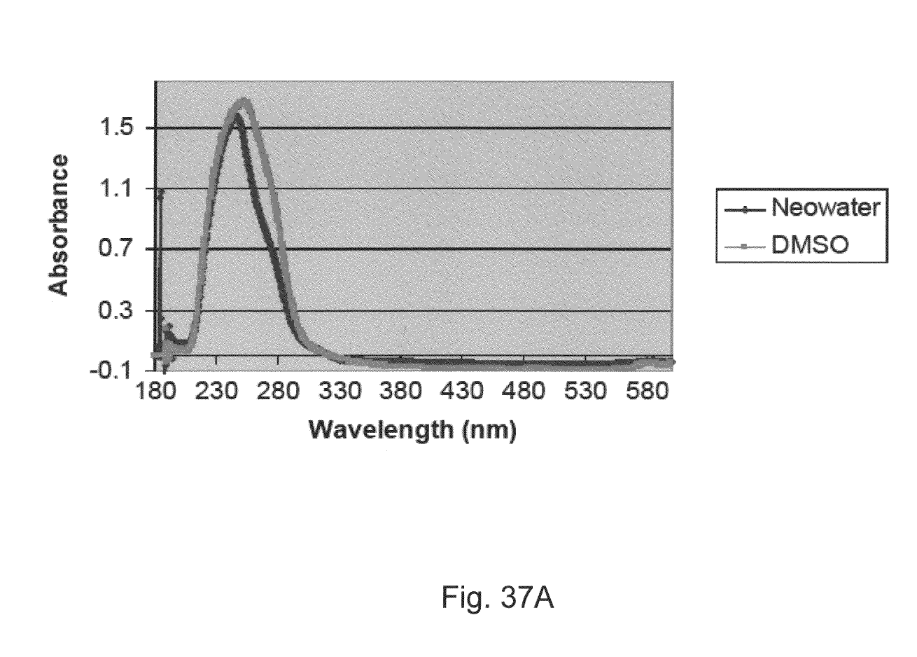

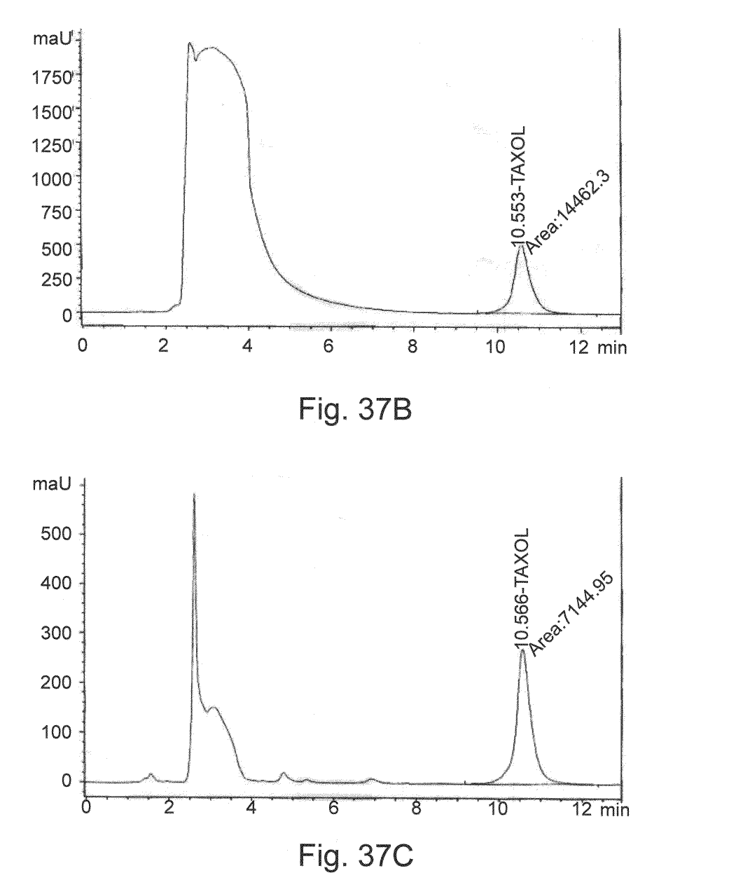

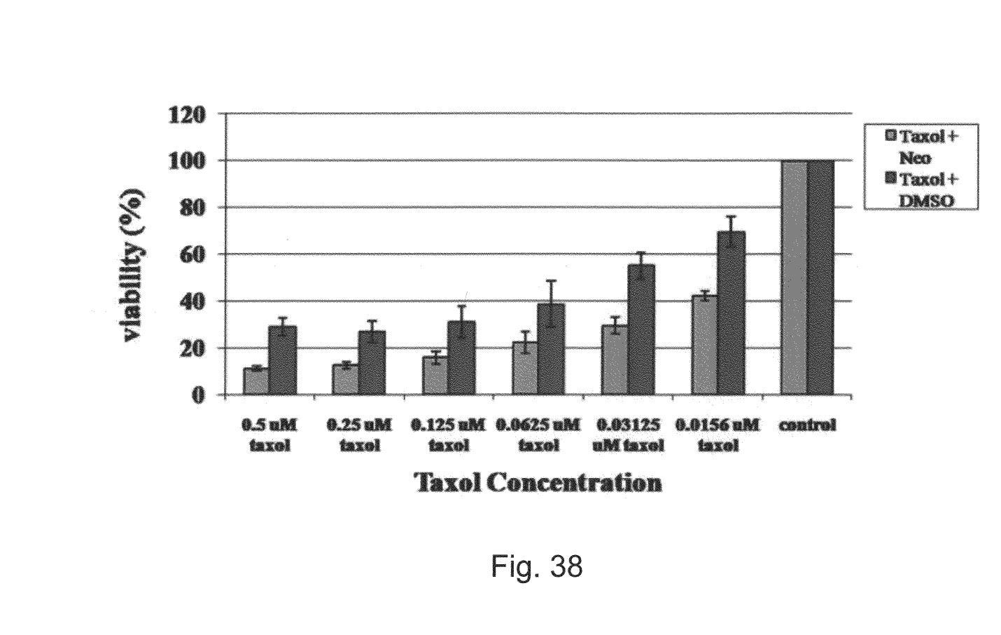

The spectrophotmetric absorbance of 0.5 mM taxol dissolved in DMSO or Neowater™ is illustrated in FIG. 37A. FIGS. 37B-C are HPLC readouts for both formulations. Measurements showed no structural changes in the formulation of taxol dispersed in Neowater™ following a 6 month storage period. The results of taxol-induced loss of cell viability is illustrated in FIG. 38 following dissolving in DMSO or Neowater™.

Conclusion

Taxol dissolved in Neowater (with 0.08% ethanol in the final working concentration) showed similar in vitro cell viability/cytotoxicity on a human prostate cancer cell line as taxol dissolved in DMSO.

### Example 17

### Cephalosporin Solubilization

The aim of the following experiments was to dissolve insoluble Cephalosporin in Neowater (NW) at a concentration of 3.6 mg/ml, using a slow solvent exchange procedure and to assess its bioactivity on E. Coli DH5α strain transformed with the Ampicillin (Amp) resistant bearing pUC19 plasmid.

Materials and Methods

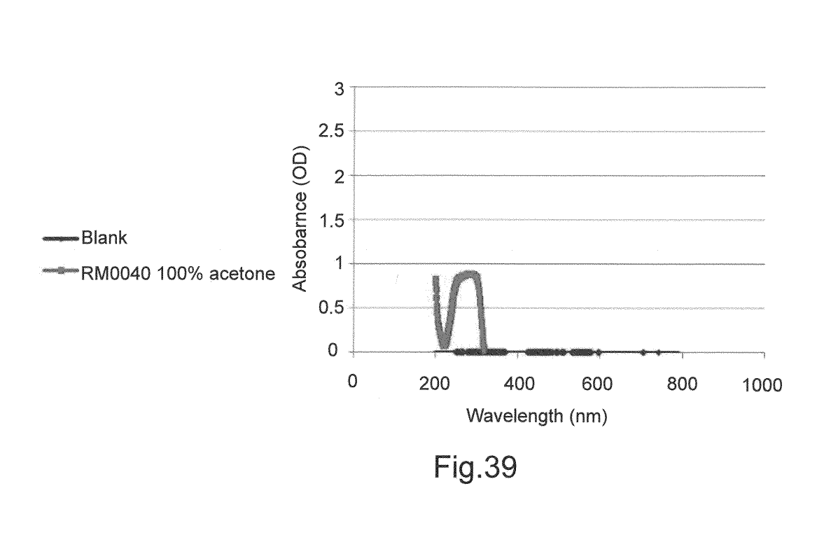

Slow solvent exchange: 25 mg of cephalosporin was dissolved in 5 ml organic solvent Acetone (5 mg/ml). Prior to addition of NW, the material was analyzed with a Heλios α spectrophotometer (FIG. 39). The material barely dissolved in acetone. It initially sedimented with a sand-like appearance. The procedure of exchanging the organic solvents with Neowater was performed on a multi block heater (set at 30° C.), and inside a desiccator and a hood. Organic solvent concentration was calculated according to the equations set forth in Table 2.

TABLE 2

Analytical Balance
% Acetone ml 1 − 0.1739X = Weighed value
% EtOH ml 1 − 0.2155X = Weighed value
Refractometer
% Acetone ml 0.0006X + 1.3328 = Refractive Index (RI) value
% EtOH ml 0.0006X + 1.3327 = Refractive Index (RI) value

Refractometer: RI: 1.3339, according to the equation calculations: 1.833%.

Analytical balance: average: 0.9962, according to the equation: 1.941%.

The solution was filtered successfully using a 0.45 μm filter. Spectrophotometer readouts of the solution were performed before and after the filtration procedure.

Analysis of bioactivity of Cephalosporin dissolved in Neowater™: DH5α E. Coli bearing the pUC19 plasmid (Ampicillin resistant) were grown in liquid LB medium supplemented with 100 μg/ml ampicillin overnight at 37° C. and 220 rpm (Rounds per minute).

100 μL of the overnight (ON) starter re-inoculated in fresh liquid LB as follows:

a. 3 tubes with 100 μl of Neowater™: (only 2^{nd} experiment) and no antibiotics (both experiments).

b. 3 tubes with 10 μl of the Cephalosporin stock solution (50 ug/ml).

c. 3 tubes with 100 μl of the Cephalosporin stock solution (5 ug/ml).

Bacteria were incubated at 37° C. and 220 rpm. Sequential OD readings took place every hour using a 96 wells transparent plate with a 590 nm filter using the TECAN SPECTRAFlour Plus.

Results

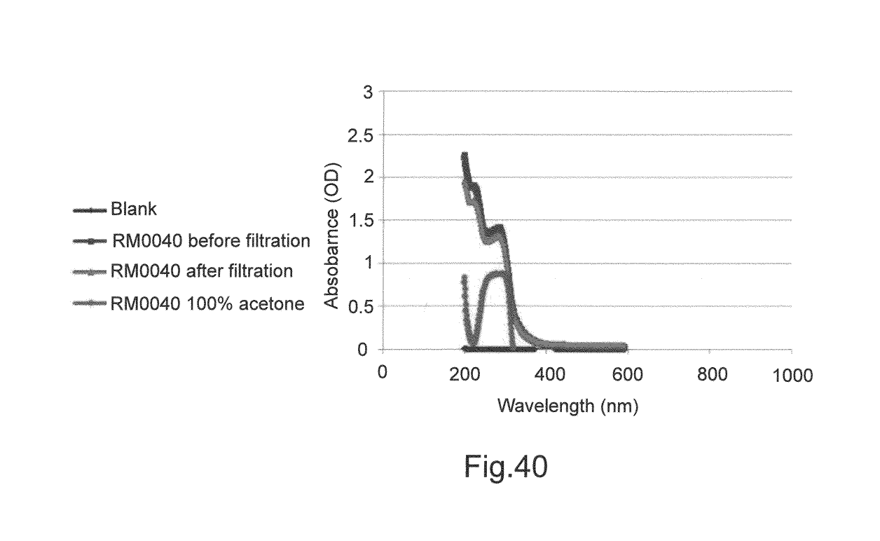

FIG. 40 is a spectrophotometer readout of Cephalosporin dissolved in Neowater™ prior to and following filtration.

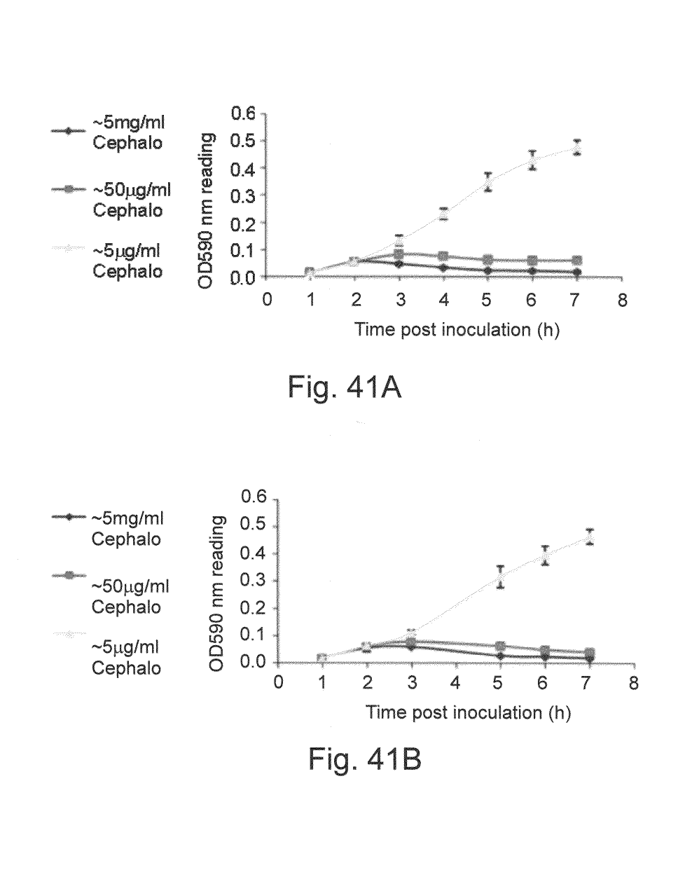

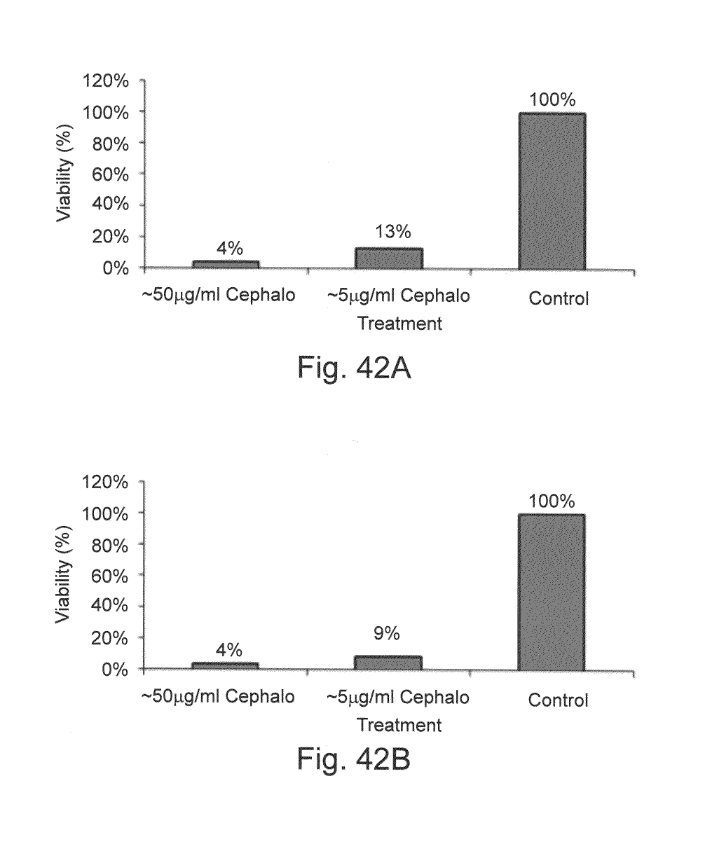

As illustrated in FIGS. 41A-B and 42A-B, when dissolved in Neowater™, Cephalosporin is bioavailable and bioactive as a bacterial growth inhibitor even when massively diluted. Of note, the present example teaches that Neowater™ itself has no role in bacterial growth inhibition.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A kit for detecting an analyte comprising:
(i) a detectable agent; and
(ii) a liquid composition having a liquid and nanostructures, each of said nanostructures comprising a core material of a nanometric size surrounded by an envelope of ordered fluid molecules, said core material and said envelope of ordered fluid molecules being in a steady physical state, wherein said nanostructures are capable of maintaining long range interaction thereamongst.

2. The kit of claim 1, wherein the analyte is a biomolecule.

3. The kit of claim 2, wherein said biomolecule is selected from the group consisting of a polypeptide, a polynucleotide, a carbohydrate, a lipid and a combination thereof.

4. The kit of claim 1, wherein said detectable agent is non-directly detectable.

5. The kit of claim 4, wherein said non-directly detectable agent is a substrate for an enzymatic reaction capable of generating a detectable product.

6. The kit of claim 1, wherein said detectable agent is directly detectable.

7. The kit of claim 1, wherein said detectable agent comprise an affinity recognition moiety.

8. (canceled)

9. The kit of claim 6, wherein said directly detectable agent is selected from the group consisting of a phosphorescent agent, a chemiluminescent agent and a fluorescent agent.

10. The kit of claim 5, further comprising an enhancer of said enzymatic reaction.

11. (canceled)

12. The kit of claim 5, further comprising an oxidizing agent.

13. (canceled)

14. The kit of claim 5, further comprising an enzyme for said enzymatic reaction.

15-16. (canceled)

17. The kit of claim 5, further comprising an inhibitor of said enzymatic reaction.

18. The kit of claim 5, wherein said detectable product is selected from the group consisting of a fluorescent product, a chemiluminescent product, a phosphorescent product and a chromogenic product.

19-23. (canceled)

24. The kit of claim 1, wherein at least a portion of said fluid molecules are identical to molecule of said liquid.

25. The kit of claim 1, wherein said at least a portion of said fluid molecules are in a gaseous state.

26. The kit of claim 1, wherein a concentration of said nanostructures is lower than 10²⁰ nanostructures per liter.

27. The kit of claim 1, wherein said nanostructures are capable of forming clusters of said nanostructures.

28. The kit of claim 1, wherein said liquid composition comprises a buffering capacity greater than a buffering capacity of water.

29. The kit of claim 1, wherein said liquid composition is formulated from hydroxyapatite.

30. An article of manufacture comprising packaging material and a liquid composition identified for enhancing detection of a detectable moiety being contained within said packaging material, said liquid composition having a liquid and nanostructures, each of said nanostructures comprising a core material of a nanometric size surrounded by an envelope of ordered fluid molecules, said core material and said envelope of ordered fluid molecules being in a steady physical state, wherein said nanostructures are capable of maintaining long range interaction thereamongst.

31. The article of manufacture of claim 30, wherein said detectable moiety is selected from the group consisting of a fluorescent moiety, a chemiluminescent moiety and a phosphorescent moiety.

32-37. (canceled)

38. A method of dissolving or dispersing cephalosporin comprising contacting the cephalosporin with nanostructures and liquid under conditions which allow dispersion or dissolving of the substance, wherein said nanostructures comprise a core material of a nanometric size enveloped by ordered fluid molecules of said liquid, said core material and said envelope of ordered fluid molecules being in a steady physical state.
